(19) United States
(12) Patent Application Publication
(10) Pub. No.: US 2025/0128056 A1
(43) Pub. Date: Apr. 24, 2025

(54) INTRAVASCULAR SCAFFOLD

(71) Applicant: Covidien LP, Mansfield, MA (US)

(72) Inventors: Eric C. Mintz, Newport Beach, CA (US); Varun Umesh Kashyap, Palo Alto, CA (US); Mark Ashby, Laguna Niguel, CA (US)

(21) Appl. No.: 18/919,802

(22) Filed: Oct. 18, 2024

Related U.S. Application Data
(60) Provisional application No. 63/591,347, filed on Oct. 18, 2023.

(51) Int. Cl. A61N 1/05; A61N 1/36

(52) U.S. Cl. CPC A61N 1/0539; A61N 1/0534; A61N 1/0536; A61N 1/3614; A61N 2001/058

(57) ABSTRACT

In some examples, an endovascular system includes an elongated body configured to be introduced into a blood vessel of a patient, a plurality of electrodes carried by the elongated body, and an elongated scaffold configured to be introduced into the blood vessel and configured to transform between a relatively low-profile delivery configuration and a deployed configuration. In the deployed configuration, the elongated scaffold is configured to exert a radial force to urge the elongated body and the plurality of electrodes towards a wall of the blood vessel. After being introduced into the blood vessel of the patient, the elongated scaffold is configured to resorb or degrade over a period of time. A degradation profile of the elongated scaffold over the period of time corresponds to a level of endothelization proximate the plurality of electrodes.

### CROSS-REFERENCE TO RELATED APPLICATIONS

This application claims the benefit of and priority to U.S. Provisional Patent Application Ser. No. 63/591,347 filed Oct. 18, 2023, the entire disclosure of which is incorporated by reference herein.

### TECHNICAL FIELD

This disclosure relates to electrical stimulation therapy.

### BACKGROUND

Medical devices, such as electrical stimulation devices, may be used in different therapeutic applications, such as deep brain stimulation (DBS) or peripheral nerve stimulation (PNS). A medical device may be used to deliver therapy to a patient to treat a variety of symptoms or patient conditions such as, but not limited to, movement disorders, seizure disorders (e.g., epilepsy), or mood disorders. In some therapy systems, an implantable electrical stimulator delivers electrical stimulation therapy to a target tissue site within a patient with the aid of one or more electrodes.

### SUMMARY

This disclosure describes example endovascular devices and systems configured to deliver electrical stimulation therapy to a brain and/or peripheral nerves of a patient and/or sense one or more patient parameters (e.g., brain signals, evoked potentials, etc.) from an endovascular location. The endovascular devices and systems described herein are configured to be relatively minimally invasive at least because they are configured to be navigated through vasculature of a patient to a blood vessel (e.g., a venous vessel or an arterial vessel) in order to deliver electrical stimulation therapy to a target tissue site (e.g., in the brain) of a patient and/or sense one or more patient parameters. After being navigated through the vasculature of the patient, a portion of a device is configured to be incorporated into a vessel wall, e.g., through endothelization. One or more structures (e.g., a scaffold) may hold portions of the device in apposition with the vessel wall. The structures that hold portions of the device in apposition with the vessel wall may degrade or resorb over time, such that only the elements configured to delivery therapy remain.

In examples described herein, an endovascular system includes an endovascular device including an elongated body and one or more electrodes carried by the elongated body. The endovascular system further includes an elongated scaffold configured to exert a radial force to urge the elongated body and the plurality of electrodes towards a wall of the blood vessel (e.g., to hold the elongated body and/or the plurality of electrodes in apposition with the blood vessel wall). Urging the elongated body and the plurality of electrodes towards a wall of the blood vessel may reduce risk of thrombosis and promote endothelization of the plurality of electrodes (e.g., such that the elongated body and the plurality of electrodes become incorporated into the vessel wall). By becoming incorporated into the vessel wall, the portion of the device configured to deliver electrical stimulation therapy or sense one or more patient parameters may do some more effectively or efficiently (e.g., by reducing the overall power needed to deliver efficacious electrical stimulation therapy).

In examples described herein, structures (e.g., scaffolds) may be configured to degrade or resorb over a period of time, such that the structures no longer remain in the vasculature and naturally break down within a patient's body. While permanent stents or other expandable structures may similarly hold devices in opposition with a vessel wall, such permanent devices may occlude or otherwise disrupt blood flow in the vasculature (e.g., neurovasculature and/or peripheral vasculature), and can lead to undesirable thrombus formation, particularly in small blood vessels, if left implanted for extended periods of time. Additionally, permanent structures such as stents may need to come in a variety of sizes (e.g., diameters and lengths) and require sufficiently large delivery systems, which may prevent such systems being used in difficult to reach or small intravascular sights. Further, other biodegradable or bioresorbable structures may last in the vasculature longer than needed, and the degradation and/or profile (e.g., cycle, time, etc.) does not otherwise correspond to the endothelization cycle of electrodes or endovascular device. On the other hand, biodegradable or bioresorbable structures that degrade too quickly, or degrade in a manner that fails to support the electrodes for a sufficient amount of time in the endothelization cycle, may result in poor endothelization of the electrodes. The scaffolds described herein may include a degradation profile that corresponds to a level of endothelization of the elongated body and/or plurality of electrodes such that the scaffolds remain in the vasculature and exert a radial force sufficiently long to promote endothelization, but do not remain in the vasculature longer than needed.

In some examples, an endovascular system includes an elongated body configured to be introduced into a blood vessel of a patient, a plurality of electrodes carried by the elongated body, and an elongated scaffold configured to be introduced into the blood vessel and configured to transform between a relatively low-profile delivery configuration and a deployed configuration. In the deployed configuration, the elongated scaffold is configured to exert a radial force to urge the elongated body and the plurality of electrodes towards a wall of the blood vessel. After being introduced into the blood vessel of the patient, the elongated scaffold is configured to resorb or degrade over a period of time. A degradation profile of the elongated scaffold over the period of time corresponds to a level of endothelization proximate the plurality of electrodes.

In some examples, a method includes inserting, into a blood vessel of a patient, an elongated body and a plurality of electrodes carried by the elongated body and inserting, into the blood vessel of the patient, an elongated scaffold, the elongated scaffold configured to transform between a relatively low-profile delivery configuration and a deployed configuration. In the deployed configuration, the elongated scaffold exerts a radial force to urge the elongated body and the plurality of electrodes towards a wall of the blood vessel. After being introduced into the blood vessel of the patient, the elongated scaffold resorbs or degrades over a period of time. A degradation profile of the elongated scaffold over the period of time corresponds to a level of endothelization proximate the plurality of electrodes.

The examples described herein may be combined in any permutation or combination.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system including an endovascular device configured to deliver electrical stimulation therapy to a target tissue site in a brain of a patient and/or sense one or more patient parameters.

FIG. 2 is a functional block diagram illustrating components of an example endovascular device of the therapy system of FIG. 1.

FIG. 3A is a side view of an example distal portion of an elongated body of an example endovascular device.

FIG. 3B is a side view of an example distal portion of an elongated body of an example endovascular device and an elongated scaffold.

FIG. 3C is a side view of an example distal portion of an elongated body of an example endovascular device and an elongated scaffold.

FIG. 3D is a side view of an example distal portion of an elongated body of an example endovascular device at least partially incorporated into a vessel wall via endothelization.

FIG. 4A is a side view of an example distal portion of an elongated body of an example endovascular device.

FIG. 4B is a side view of an example distal portion of an elongated body of an example endovascular device and an elongated scaffold.

FIG. 5A is a side view of an example distal portion of an elongated body of an example endovascular device.

FIG. 5B is a side view of an example distal portion of an elongated body of an example endovascular device and an elongated scaffold.

FIG. 6A illustrates a section view of a strut or braid of an example elongated scaffold.

FIG. 6B illustrates a section view of a strut or braid of an example elongated scaffold including a first material and a second material.

FIG. 6C illustrates a section view of a strut or braid of an example elongated scaffold.

FIG. 7A is a graph of a degradation profile of an example elongated scaffold.

FIG. 7B is a graph of a degradation profile of an example elongated scaffold including at least a first material and a second material.

FIG. 8A is a graph of a level of endothelization versus time of a portion of an elongated body and/or electrodes.

FIG. 8B is a graph of a degradation profile of an example elongated scaffold.

FIG. 8C is a graph of a force exerted by an example elongated scaffold versus time corresponding to the time of FIG. 8A.

FIG. 9A illustrates a side view of an example elongated scaffold including struts and/or braid portions of different sizes.

FIG. 9B illustrates a side view of an example elongated scaffold including varied spacing of struts and/or braid potions.

FIG. 10 illustrates a side view of an example elongated scaffold including an example elongated body interwoven through the elongated scaffold.

FIG. 11 is a flow chart illustrating an example method of delivering an example endovascular device and an elongated scaffold.

Like reference characters denote like elements throughout the description and figures.

### DETAILED DESCRIPTION

This disclosure describes devices, systems, and methods relating to deep brain stimulation (DBS), such as delivery of electrical stimulation therapy to one or more deep brain stimulation (DBS) targets from an endovascular location, as well as sensing of signals from one or more endovascular locations. Example endovascular locations that can be used to access the DBS sites and/or sense of signals using the devices described herein include any suitable cranial vein or cranial artery, such as, but not limited to, the thalamostriate vein, the internal cerebral vein, the basal vein of Rosenthal, the inferior sagittal sinus, the superior sagittal sinus, or the anterior choroidal artery. Other sites of interest may include the internal jugular vein, such as for vagus nerve stimulation.

DBS has been proposed for use to manage one or more patient conditions, such as to treat a patient condition by reducing or even eliminating one or more symptoms associated with the patient condition. For example, DBS can be used to alleviate, and in some cases eliminate, symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD).

In the examples described herein, an endovascular system includes an endovascular device including an elongated body and one or more electrodes carried by the elongated body. The endovascular system further includes an elongated scaffold configured to exert a radial force to urge the elongated body and the plurality of electrodes towards a wall of the blood vessel (e.g., to hold the elongated body and/or the plurality of electrodes in apposition with the blood vessel wall). Urging the elongated body and the plurality of electrodes towards a wall of the blood vessel may reduce risk of thrombosis and promote endothelization (e.g., such that the elongated body and the plurality of electrodes become incorporated into the vessel wall). By becoming incorporated into the vessel wall, the portion of the device configured to deliver electrical stimulation therapy or sense one or more patient parameters may do some more effectively or efficiently (e.g., by reducing the overall power needed to deliver efficacious electrical stimulation therapy).

The scaffolds described herein may facilitate endothelization of the plurality of electrodes and/or the elongated body (or portions thereof) into the wall of the blood vessel. Endothelization of the plurality of electrodes and/or the elongated body may involve formation of an endothelial layer on the surface of the electrodes and/or elongated body. Endothelization may reduce the overall power needed to deliver efficacious electrical stimulation therapy to a patient, as well as help secure the electrodes in place in the blood vessel in instances of chronic therapy delivery. In examples described herein, the scaffolds are configured to exert a radial force to urge the elongated body and the plurality of electrodes towards a wall of the blood vessel (e.g., to hold the elongated body and/or the plurality of electrodes in apposition with the blood vessel wall). Urging the elongated body and the plurality of electrodes towards a wall of the blood vessel may reduce risk of thrombosis and promote endothelization (e.g., such that the elongated body and the plurality of electrodes become incorporated into the vessel wall). The scaffolds described herein may urge the electrodes toward a particular part of the vessel wall (e.g., an inner or outer part of a curve of a vessel). In some examples, the scaffold includes a tubular body and a plurality of struts, coils, or other braid-like structures (e.g., that make up the tubular body). The struts can include a single material (e.g., core material), or two or more materials that may have different rates of degradation or resorption. The struts can have uniform size, or different sizes. The struts can have uniform spacing, or the scaffolds can have groups of struts with different spacing.

In examples described herein, the structures (e.g., scaffolds) may be configured to degrade or resorb over a period of time, such that the structures no longer remain in the vasculature and break down with a patient's body. While permanent stents or other expandable structures may similarly hold devices in opposition with a vessel wall, such devices may occlude or otherwise disrupt blood flow in the neurovasculature, and can lead to undesirable thrombosis formation, particularly in small blood vessels, if left implanted for extended periods of time. Permanent stents may also impact vessel wall dynamics (sometimes referred to as “caging” a vessel) in which the permanent stent prevents normal expansion and/or contraction during the systole and diastole or distention and contraction cycles of the vessel, which can weaken and cause long-term impact to the vessel wall as well as further impact blood flow dynamics due to the loss of elasticity of the vessel wall. Additionally, permanent structures such as stents may need to come in a variety of sizes (e.g., diameters and lengths) and require large enough delivery systems, which may prevent such systems being used in difficult to reach or small intravascular sights. Further, other biodegradable or bioresorbable structures may last in the vasculature longer than needed, and the degradation does not otherwise correspond to the endothelization cycle. The scaffolds described herein may include a degradation profile that corresponds to a level of endothelization of the elongated body and/or plurality of electrodes such that the scaffolds do not remain in the vasculature longer than needed. Once the scaffolds completely degrades, only the endovascular device (which may be incorporated into a vessel wall via endothelization) remains for chronic stimulation therapy and/or sensing.

The endovascular devices described herein include an elongated body and one or more electrodes carried by, defined by, or otherwise disposed on the elongated body. Additionally, the elongated body includes a distal portion that is substantially straight (e.g., does not coil). However, in other examples, the endovascular device may include a portion that includes a coil, and may be configured to deploy (e.g., expand).

While the endovascular devices and scaffolds described herein are described primarily in the context of placement within the neurovasculature, it should be appreciated that the techniques of this disclosure can be used for placing devices and scaffolds in other places. For example, the devices and techniques described herein may be configured for placement in one or more of a dural sinus, vein, or artery in close proximity to a target area in the brain, but may also be adapted and configured for placement in the peripheral vasculature in proximity to a peripheral nerve for electrical stimulation therapy and/or sensing. In some examples, the devices and techniques of this disclosure may also be adapted and configured for placement within the ventricles or other hollow anatomical structures of the brain.

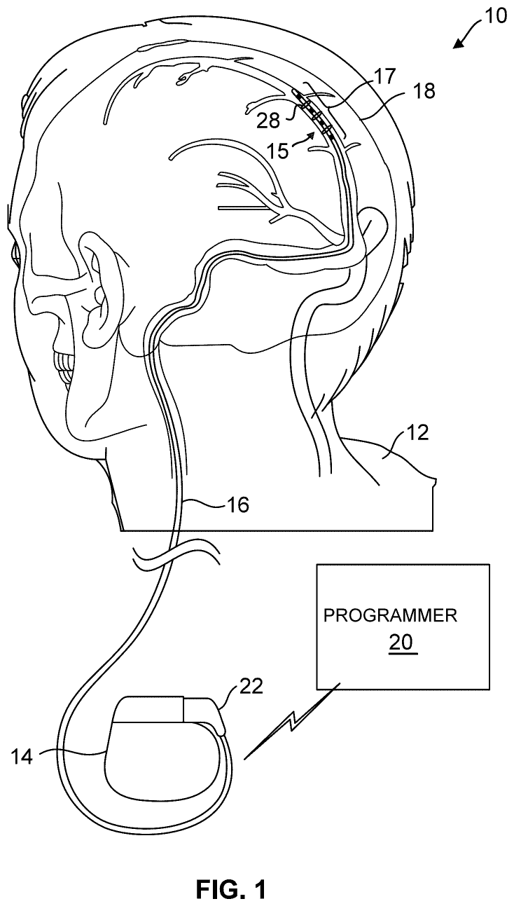

FIG. 1 is a conceptual diagram illustrating an example endovascular therapy system 10 (also referred to herein as therapy system 10 or endovascular system 10) configured to deliver electrical stimulation therapy to a target tissue site in a brain 18 of a patient 12. Patient 12 ordinarily will be a human patient. In some cases, however, therapy system 10 may be applied to other mammalian or non-mammalian non-human patients. In some examples, therapy system 10 includes medical device 14, an endovascular device 16, a scaffold 28 (which may also be referred to herein as elongated scaffold 28) and a plurality of electrodes 17 disposed on a distal portion 15 of endovascular device 16. In the example shown in FIG. 1, medical device 14 is configured to deliver DBS to brain 18 of patient 12 and/or sense bioelectrical brain signals in brain 18 via plurality of electrodes 17 of endovascular device 16. Endovascular device 16 is positioned in cranial vasculature of patient 12, such that plurality of electrodes 17 are located proximate to a target tissue site within brain 18 and are positioned to deliver electrical stimulation therapy to brain tissue sites within brain 18 and/or sense one or more patient parameters from the brain tissue sites, such as tissue sites under the dura mater surrounding brain 18. In some examples, placement of endovascular device 16, distal portion 15, and plurality of electrodes 17 is coincident with the dura mater, such as in the middle meningeal artery (MMA). Medical device 14 can provide electrical stimulation to one or more regions within brain 18 in order to manage a condition of patient 12, such as to mitigate the severity or duration of the patient condition, and/or sense one or more patient parameters to provide data and/or feedback required for managing a condition of the patient 12.

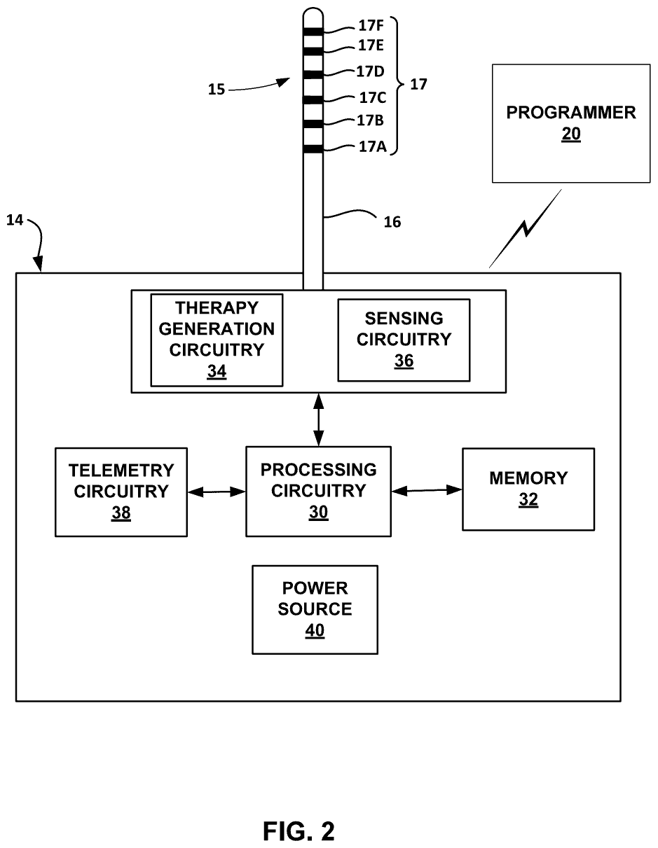

Endovascular device 16 includes any elongated body configured to deliver electrical stimulation signals to, and/or sense one or more patient parameters from, tissue proximate plurality of electrodes 17. For example, endovascular device 16 can be a medical lead, a catheter, a guidewire, or another elongated body carrying plurality of electrodes 17 and configured to be electrically coupled to medical device 14 either directly or indirectly via an electrically conductive pathway that runs between medical device 14 and plurality of electrodes 17. The elongated body of endovascular device 16 is configured to be introduced into a blood vessel of patient 12. Endovascular device 16 has any suitable length that enables connection to medical device 14 either directly or indirectly, e.g., a length of 150 centimeters (cm) to 250 cm, such as 200 cm. As another example, endovascular device 16 can be a wireless therapy delivery device, such as a microstimulator or the like, which is not electrically coupled to medical device 14 via a wired connection. In some of these wireless therapy delivery device examples, system 10 does not include medical device 14 and endovascular device 16 includes therapy generation circuitry and/or other elements of medical device 14 described herein, e.g., with respect to FIG. 2. In some of these wireless therapy delivery device examples, distal portion 15 including plurality of electrodes 17 is configured to detach (e.g., via a detachment mechanism) from an elongated deliver member (e.g., a push wire or a hypotube) used to deliver distal portion 15 to a target site.

In some examples, more than one endovascular device 16 may be positioned within brain 18 of patient 12 to provide stimulation to, and/or sense one or more patient parameters from, multiple anatomical regions of brain 18. Endovascular device 16 can be implanted in a blood vessel for chronic therapy delivery (e.g., on the order of months or even years) or for more temporary therapy delivery (e.g., on the order of days, such as less than a month or less than 6 months). In some examples, one or more devices (e.g., one or more of endovascular device 16) are placed to provide stimulation and/or sense in corresponding regions of the brain, such as in the cortex. In some examples, one or more endovascular devices are placed within intracranial venous structures to provide electrical stimulation and/or sense in corresponding regions of the brain. When endovascular devices are placed in different regions of the brain, for example within multiple arterial locations, multiple venous locations, or within arterial and venous locations (e.g., MMA and deep venous system), the combined sensing from both modalities may provide temporal and spatial data. The temporal and spatial data can be used to control delivery of electrical stimulation therapy to patient 12 and/or to evaluate a patient condition at one point in time or over a longer time period.

DBS may be used to treat various patient conditions, such as, but not limited to, seizure disorders (e.g., epilepsy), pain, migraine headaches, psychiatric disorders (e.g., obsessive compulsive disorder, mood disorders or anxiety disorders), movement disorders (e.g., essential tremor or Parkinson's disease), Huntington's disease, and other neurodegenerative disorders. The anatomic region within brain 18 of patient 12 that serve as the target tissue site for electrical stimulation delivered by medical device 14 may be selected based on the patient condition. For example, stimulating an anatomical region, such as the substantia nigra, in brain 18 may reduce the number and magnitude of tremors experienced by patient 12. Other example target anatomical regions for treatment of movement disorders may include the subthalamic nucleus, globus pallidus interna, ventral intermediate, and zona inserta. Anatomical regions such as these may be targeted by the clinician during implantation of endovascular device 16. In other words, the clinician may attempt to position endovascular device 16 within or proximate to these target regions within brain 18 by positioning endovascular device 16 in a cranial blood vessel that is within or proximate to these target regions.

In various examples described herein, example regions of brain 18 that can include the target tissue site for electrical stimulation or sensing via endovascular device 16 positioned in a blood vessel in brain 18 include, but are not limited to, one or more of the anterior thalamus, the ventrolateral thalamus, the subthalamic nucleus (STN), the substantia nigra pars reticulata, the internal segment and/or external segments of the globus pallidus, the ventral intermediate, the zona inserta, the hippocampus (HIP), the dentate gyrus, the cortex (e.g., the motor strip, the sensor strip, the premotor cortex), the fornix, the neostriatum, the ventral intermediate nucleus of the thalamus, the cingulate, or the cingulate gyrus.

The vasculature into which endovascular device 16 may be inserted and/or guided includes, but is not limited to, veins or arteries. For example, to reach certain deep brain tissue sites, endovascular device 16 can be navigated from a vasculature access site (e.g., in the femoral artery, the radial artery, femoral vein, subclavian vein, internal jugular vein or another suitable access site) to one or more arterial structures (including, but not limited to the MMA) or veins of the superficial and a deep venous system (including, but not limited to the thalamostriate vein, the internal cerebral vein, the basal vein of Rosenthal, the inferior/superior sagittal sinus, the anterior choroidal artery, or any related combinations thereof).

Certain intracranial blood vessels into which endovascular device 16 may be inserted and/or guided may be located at different distances from different target tissue sites. Such distances may play a role in efficacy of therapy delivered by endovascular device 16, as a closer distance may indicate a shorter distance any electrical stimulation signal may have to travel, and, in some examples, the less power that is needed to generate an efficacious electrical stimulation signal. For example, the thalamostriate vein may be approximately 1.2 millimeters (mm) in diameter and be located approximately 0-2 mm from the anterior nucleus of the thalamus (ANT) and 0-2 mm from the fornix. As another example, the internal cerebral vein may be 1.9 mm plus or minus up to 0.5 mm in diameter and be located approximately 5-10 mm from the ANT and approximately 2-5 mm from the fornix. The basal vein of Rosenthal may be 1.7 mm plus or minus up to 0.4 mm in diameter and be located approximately 10-15 mm from the ANT, approximately 5-10 mm from the HIP, and approximately 5-10 mm from the STN. The inferior sagittal sinus may be 1.3 mm plus or minus up to 0.3 mm in diameter and be located approximately 10-15 mm from the Fornix.

A clinician can also select a particular intracranial blood vessel to position plurality of electrodes 17 at different orientations or distances relative to tissue sites (along with selectively activating groups of electrodes that face a certain direction) for which it may be desirable to avoid electrical stimulation to minimize or even eliminate adverse effects. DBS may cause one or more side effects by inadvertently providing electrical stimulation to anatomical regions near the targeted anatomical region. For this reason, a clinician may position plurality of electrodes 17 within brain 18 and/or program the electrical stimulation parameters in order to balance effective therapy and minimal side effects.

As discussed in further detail below, in some examples, endovascular device 16 is configured to be delivered to one or more target sites in brain 18 via vasculature of patient 12. Thus, rather than introducing endovascular device 16 into brain tissue (e.g., the cerebral parenchyma) via a burr hole through a skull of patient 12 or the like, endovascular device 16 is configured to be navigated to a target electrical stimulation site in brain 18 via vasculature of patient 12. The endovascular delivery of endovascular device 16 to deep brain sites in brain 18 can help minimize the invasiveness of therapy system 10.

In some examples, as discussed below, plurality of electrodes 17 are positioned on a portion of endovascular device 16 that may be configured to deploy (e.g., expand) radially outwards from a relatively low-profile delivery configuration to a deployed (e.g., coiled or expanded) configuration. This may enable plurality of electrodes 17 to be held in apposition with a blood vessel wall, which may promote tissue ingrowth or endothelization around plurality of electrodes 17 along the vessel wall (while still maintaining a vessel lumen). Tissue ingrowth or endothelization around plurality of electrodes 17 can reduce the overall power needed to deliver efficacious electrical stimulation therapy to a target tissue site of brain 18, and help secure plurality of electrodes 17 in place in the blood vessel for chronic therapy delivery.

In some examples, at least distal portion 15 of endovascular device 16 includes a shape memory material (e.g., nitinol) material that enables distal portion 15 to assume a predetermined shape in the absence of a force (e.g., a compressive or tensile force) holding the distal portion 15 in a relatively low-profile delivery configuration. For example, distal portion 15 can be configured to expand radially outwards upon deployment from an outer sheath (e.g., an outer catheter), or upon the proximal withdrawal of a straightening element (e.g., a guidewire or a mandrel) positioned in an inner lumen of endovascular device 16. As another example, the distal portion 15 can be configured to expand radially outwards in response to proximal withdrawal of a pull member attached to distal portion 15 of the endovascular device 16 or in response to a distal movement of an elongated control member attached to distal portion 15.

In some examples, as discussed below, plurality of electrodes 17 are positioned on a portion of endovascular device 16 that does not radially expand outwards. For example, plurality of electrodes 17 may be positioned on a portion of endovascular device 16 that is straight, substantially straight, and/or that conforms to the vasculature when implanted in patient 12.

Electrodes 17 can have any suitable configuration and arrangement, including a segmented or partial ring configuration. In some examples, some or all of plurality of electrodes 17 are integrally formed with at least distal portion 15 of endovascular device 16. For example, at least distal portion 15 of endovascular device 16 is formed from an electrically conductive material (e.g., one or more nitinol core wires) that is electrically connected to therapy generation circuitry 34 and sensing circuitry 36 (FIG. 2) and an electrically insulative material can be positioned radially outwards of the electrically conductive material to cover the electrically conductive material. In such examples, where endovascular device 16 includes an electrically insulative material, at least a portion of the electrically insulative material is removed to expose the electrically conductive material to define plurality of electrodes 17. To define plurality of electrodes 17, part of the electrically insulative material can be removed (e.g., via laser ablation, mechanical etching, or the like) to expose the electrically conductive material. Any suitable electrically insulative material can be used, such as, but not limited to, polytetrafluoroethylene (PTFE), expanded PTFE (ePTFE), silicone, polyimide, non-metallic oxide, parylene or the like. The electrically insulative material can have any suitable thickness, such as, but not limited to, 0.010 mm to 0.05 mm (e.g., about 0.0005 inches).

In other examples, plurality of electrodes 17 are or include a component physically separate from endovascular device 16 and mechanically connected to the endovascular device 16. For example, plurality of electrodes 17 includes an electrically conductive electrode material (e.g., platinum, tungsten, gold, or the like, which can be radiopaque or not) electrically coupled to one or more electrically conduct materials extending through endovascular device 16.

In some examples, as discussed below, elongated scaffold 28 is configured to be introduced into a blood vessel and configured to exert a radial force (e.g., radially outward from a longitudinal axis of scaffold 28 and/or radially outward from a radial center of the blood vessel) on a portion of endovascular device 16 (e.g., the elongated body of endovascular device 16) and/or plurality of electrodes 17 to urge the portion of endovascular device 16 and/or plurality of electrodes 17 towards a wall of the blood vessel. Urging the portion endovascular device 16 and/or plurality of electrodes 17 towards a wall of the blood vessel may promote endothelization. As discussed elsewhere, endothelization may reduce the overall power needed to deliver efficacious electrical stimulation therapy to a patient, as well as help secure the electrodes in place in the blood vessel in instances of chronic therapy delivery. Additionally, the risk of thrombosis otherwise caused by occlusive structures within the neurovasculature may be reduced, e.g., in response to endothelization.

In some examples, scaffold 28 is configured to transform between a relatively low-profile delivery configuration and a deployed configuration. In the low-profile delivery configuration, scaffold 28 may be configured to be collapsed to a smaller profile (e.g., having a smaller maximum radial dimension) and loaded into a delivery device, which may be navigated to a target location with the vasculature. In the deployed configured, scaffold may be configured to radially expand outward, such as to provide a radially outward force (e.g., to urge the elongated body of endovascular device 16 and plurality of electrodes 17 towards a wall of the blood vessel). In some examples, scaffold 28 is self-expanding or partially self-expanding. In some examples, scaffold 28 is expanded (or at least partially expanded) via a suitable expansion mechanism (e.g., by a balloon, via a pullwire, via electrical energy, via thermal energy, etc.). As described below, scaffold 28 may include a suitable structure, material, or combination thereof to exert a radial force outward.

As discussed below, in some examples, scaffold 28 is configured to resorb or degrade over a period of time (e.g., once implanted within patient 12). In this way, scaffold 28 may be configured to urge a portion of endovascular device 16 and/or plurality of electrodes 17 against a vessel wall for a period of time, and subsequently resorb or degrade. After the portion of endovascular device 16 and/or plurality of electrodes 17 has reached a sufficient level of endothelization into the vessel wall (e.g., a level such of incorporation into the vessel wall such that a threshold impedance, power, or other parameter has been reached), the radial force of scaffold 28 may no longer be needed to promote endothelization.

In some examples, a degradation profile (e.g., reduction of material over time, rate of degradation, etc.) of scaffold 28 corresponds to a level of endothelization proximate plurality of electrodes 17. For example, scaffold 28 may be configured to degrade such that scaffold 28 exerts at least a threshold force (e.g., to urge endovascular device 16 and/or plurality of electrodes 17 against a wall of a blood vessel to maintain contact of plurality of electrodes 17 with the wall of the blood vessel) for a period of time. In some examples, the degradation profile of scaffold 28 is based on one or more of a thickness of scaffold 28 (e.g., a thickness of a wall of the tubular body of scaffold 28 and/or a thickness of individual struts of scaffold 28), a material of scaffold 28, an arrangement of materials within scaffold 28, or the like. Scaffold 28 may be configured to not only remain intact until a sufficient level of endothelization has been reached, but may be configured to provide at least the threshold force until the sufficient level of endothelization has been reached. By coordinating the degradation profile of scaffold 28 with the time for sufficient endothelization of endovascular device 16 and/or plurality of electrodes 17, scaffold 28 does not remain in the vasculature longer than necessary while still promoting endothelization. By not remaining in the vasculature longer than necessary, scaffold 28 may reduce a risk of thrombus or other complication that may be a problem with other structures (e.g., permanent structures) in the vasculature.

In addition to corresponding to time period to achieve sufficient endothelization of endovascular device 16 and/or plurality of electrodes 17, the degradation profile of scaffold 28 may correspond to (e.g., be selected or tuned to) one or more patient parameters. Various physiological factor may affect the degradation profile of scaffold 28, including one or more patient factors, which may include one or more of a biological indicator (e.g., age, gender, etc.), a disease state (e.g., a disease that may otherwise affect blood composition, viscosity, etc.), an implant location (e.g., the specific blood vessel with in the neurovasculature), a blood vessel size (e.g., inner diameter), a blood vessel type (e.g., a vein versus an artery, as blood flow characteristics or vessel wall properties may differ between a vein and an artery), a blood pressure (e.g., which may vary from patient to patient, or even between time periods for a given patient), and/or a blood velocity. For example, a higher blood velocity and/or pressure (whether due to implant location or inherently in the patient) may affect the degradation profile of scaffold 28, such that scaffold 28 resorbs or degrades more quickly. In some examples, the degradation profile of scaffold 28 may be configured (e.g., selected based on or tuned to account for) one or more patient factors. In some examples, multiple scaffolds may be provided in a system (e.g., in a kit), wherein each scaffold has a different degradation or resorption profile, and a particular scaffold may be selected (e.g., by a clinician) based on the one or more patient factors noted above.

In some examples, scaffold 28 is configured to maintain contact of a portion of endovascular device 16 and/or plurality of electrodes 17 with a blood vessel wall for a period of time (e.g., until sufficient endothelization has been achieved). By maintaining contact with the blood vessel wall, the portion of endovascular device 16 and/or plurality of electrodes 17 may become incorporated via endothelization into the blood vessel wall. For example, endothelial cells may migrate from the vessel wall and adhere to portions of the endovascular device 16 and/or plurality of electrode 17 that are in contact with or proximate to the vessel wall, such that maintaining contact with the vessel wall may promote such migration and adhesion. Further, by maintaining contact with the blood vessel wall, the risk of thrombus formation by the portion of endovascular device 16 and/or plurality of electrodes 17 may be reduced. In some examples, the period of time (or a portion thereof) where elongated scaffold 28 maintains contact of plurality of electrodes 17 with the wall of the blood vessel corresponds to a threshold level of endothelization proximate plurality of electrodes 17. In some examples, scaffold 28 is configured to exert at least a threshold force on plurality of electrodes 17 for a portion of the period of time to maintain contact of plurality of electrodes 17 with the wall of the blood vessel.

In some examples, plurality of electrodes 17 and/or endovascular device 16 can include one or more surface textures or coatings to promote endothelization, decrease impedance, reduce thrombosis, or increase longevity. Materials used may include one or more of Titanium Nitride, Platinum, or poly(3,4-ethylenedioxythiophene) polystyrene sulfonate (“PEDOT:PSS” or “PDOT”). The one or more surface textures or coatings may increase a surface area of plurality of electrodes 17, which can help stabilize the impedance over a range of frequencies (e.g., of an electrical stimulation signal).

Medical device 14 can be an external medical device or an implantable medical device that includes electrical stimulation circuitry configured to generate and deliver electrical stimulation therapy to patient 12 via plurality of electrodes 17 of endovascular device 16. Plurality of electrodes 17 may be configured to deliver electrical stimulation to tissue of brain 18 of patient 12 from a location within a blood vessel. In the example shown in FIG. 1, endovascular device 16 is coupled to medical device 14 via connector 22, which defines a plurality of electrical contacts for electrically coupling plurality of electrodes 17 to electrical stimulation generation circuitry within medical device 14. Connector 22 may also be referred to as a connector block or header of medical device 14. In some examples, endovascular device 16 is indirectly coupled to connector 22 with the aid of a lead extension. In some examples, endovascular device 16 is directly coupled to connector 22 without the aid of a lead extension.

In some examples, medical device 14 is configured to be implanted in patient 12 in any suitable location, such as a location outside of brain 18, e.g., in a pectoral region. In other examples, medical device 14 is configured to be external to patient 12. Endovascular device 16 may be, for example, implanted within a cranial vein and one or more proximal wires/leads can remain within the venous system until they exit the subclavian vein in the chest for implant in the pectoral region. In yet other examples, some or all of medical device 14 is configured to be implanted in brain 18, e.g., as part of endovascular device 16.

As shown in FIG. 1, system 10 may also include a programmer 20, which may be a handheld device, portable computer, or workstation that provides a user interface to a clinician or other user. The clinician may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include an electrode combination, a power, and an amplitude, which may be a current or voltage amplitude, and, if medical device 14 delivers electrical pulses, a pulse width, and a pulse rate or frequency for stimulation signals to be delivered to the patient. Other example therapy parameters include a slew rate, duty cycle, and phase of the electrical stimulation signal. An electrode combination may include a selected group (e.g., electrodes that face the same direction) or subset (e.g., less than all of the electrodes) of plurality of electrodes 17 located on one or more implantable elongated bodies (such as endovascular device 16) coupled to medical device 14. The electrode combination may also refer to the polarities of the electrodes in the selected subset. By selecting particular electrode combinations, a clinician may target particular anatomic structures within brain 18 of patient 12. In addition, by selecting values for slew rate, duty cycle, phase amplitude, pulse width, and/or pulse rate, the clinician can attempt to generate an efficacious therapy for patient 12 that is delivered via the selected electrode subset.

Whether programmer 20 is configured for clinician or patient use, programmer 20 may communicate with medical device 14 or any other computing device via wireless or a wired communication. Programmer 20, for example, may communicate via wireless communication with medical device 14 using radio frequency (RF) telemetry techniques known in the art. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

In some examples, in addition to or instead of delivering electrical stimulation to brain 18, endovascular device 16 can be used to sense one or more patient parameters, such as bioelectrical signals, either using plurality of electrodes 17 or other types of sensors that are carried by endovascular device 16. In some examples, a sensed patient parameter includes an impedance detected via plurality of electrodes 17. In some examples, the bioelectrical signals sensed within brain 18 reflect changes in electrical current produced by the sum of electrical potential differences across brain tissue. Examples of bioelectrical brain signals that can be sensed via one or more electrodes of plurality of electrodes 17 include, but are not limited to, electrical signals generated from local field potentials within one or more regions of brain 18, an electroencephalogram (EEG) signal, an electrocorticogram (ECoG) signal, or an evoked potential. In some examples, a sensing parameter includes one or more of a direction faced by each sensing electrode or group of electrodes (e.g., the active electrodes with which a medical device senses a patient parameter), a location of one or more electrodes within brain 18, or other parameters that may affect detection and/or sensing of one or more patient parameters.

Brain 18 in FIG. 1 is supplied with blood through the carotid and the vertebral arteries on each side of the neck. The arteries include the common carotid artery in the neck, which is a common access pathway for the various devices and/or methods disclosed herein, the internal carotid which supplies the ophthalmic artery. The external carotid supplies the maxillary artery, the middle meningeal artery (MMA), and the superficial temporal arteries (frontal and parietal). The vertebral artery supplies the basilar artery and the cerebral arteries including the posterior cerebral artery and the circle of Willis. The siphon of the vertebral artery appears in the intra-cranial vasculature on the vertebral approach to the Circle of Willis. Also supplied by the internal carotid artery are the anterior cerebral artery and the middle cerebral artery, as well as the circle of Willis, including the posterior communicating artery and the anterior communicating artery. The siphon of the internal carotid artery appears in the intra-cranial vasculature on the carotid approach into the Circle of Willis. These arteries can have an internal diameter of about 1 mm to 5 mm, most commonly from 2-4 mm.

The devices, systems, and methods described herein enable endovascular delivery to deep brain tissue sites in brain 18. Endovascular device 16 can be navigated to the cranial vasculature to reach the deep brain tissue sites, e.g., via an insertion catheter (e.g., a microcatheter). As an example, endovascular device 16 can be delivered to an intracranial blood vessel inside of a 0.017 inch (about 0.43 mm) or a 0.021 inch (about 0.53 mm) microcatheter, alone or with the aid of a guidewire.

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

FIG. 2 is a functional block diagram illustrating components of an example medical device 14, which is configured to generate and deliver electrical stimulation therapy to patient 12 and, in some examples, sense one or more patient parameters, such as bioelectrical brain signals of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate electrical stimulation signals to a target tissue site in brain 18 of patient 12. Processing circuitry 30 is configured to control therapy generation circuitry 34 to generate and deliver electrical stimulation therapy via plurality of electrodes 17 of endovascular device 16. Plurality of electrodes 17 may include a monopolar or bipolar arrangement. The electrical stimulation parameter values may be selected based on the patient condition being addressed, as well as the target tissue site in brain 18 for the electrical stimulation therapy. The electrical stimulation therapy can be provided via stimulation signals of any suitable form, such as stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from plurality of electrodes 17 configured to deliver electrical stimulation therapy. Processing circuitry 30 can use the sensed physiological signals to control therapy delivery by therapy generation circuitry 34, e.g., the timing of the therapy delivery or one or more characteristics of the electrical simulation signal generated by therapy generation circuitry 34.

In some examples, sensing circuitry 36 is configured to sense a bioelectrical brain signal via plurality of electrodes 17 (e.g., all or a subset of electrodes 17). Thus, plurality of electrodes 17 can be configured to receive or transmit energy (e.g., current). Example bioelectrical brain signals include an EEG signal, an ECoG signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger). In examples in which endovascular device 16 is configured to sense an evoked potential, endovascular device 16 may also be configured to generate a stimulus (e.g., via therapy generation circuitry, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, processing circuitry 30, alone or in combination with sensing circuitry 36, is configured to determine a level of endothelization proximate one or more of the electrodes 17 based on a sensed parameter (e.g., which may also be referred to as a patient parameter). The parameter is any suitable parameter that can be sensed by processing circuitry 30 via electrodes 17 or another sensor and that changes as a function of the level of endothelization and can include, for example, impedance of an electrical pathway that includes the one or more electrodes 17. In some examples, a level of endothelization proximate plurality of electrodes 17 includes the extent to which endothelial cells cover endovascular device 16 and/or one or more electrodes 17, which may affect the impedance of the respective electrodes 17. The level can include a level at a particular point in time or a change in a level of endothelization over time, e.g., relative to a previously determined level, such as a baseline level at the time of implantation of endovascular device 16 in patient 12. For example, the change in a level of endothelization (e.g., an altered impedance) can be a result of sensing a level of impedance of one or more electrodes of the plurality of electrodes 17 at a first timepoint and at a second time point, and calculating a change in the impedance level between the first timepoint and the second timepoint.

In some examples, processing circuitry 30, alone or in combination with therapy generation circuitry 34, is configured to select one or more electrical stimulation parameter values based on a determined level of endothelization or in response to detecting a change in the level of endothelization proximate plurality of electrodes 17. As described above, the electrical stimulation parameters (or “therapy parameters”) may include an electrode combination (e.g., selective activation of a group or subset of electrodes from plurality of electrodes 17), a power, and an amplitude, which may be a current or voltage amplitude, and, if medical device 14 delivers electrical pulses, a pulse width, and a pulse rate or frequency for stimulation signals to be delivered to the patient. Other example electrical stimulation parameters include a slew rate, duty cycle, and phase of the electrical stimulation signal. In some examples, processing circuitry 30, alone or in combination with therapy generation circuitry 34, is configured to automatically select or adjust one or more electrical stimulation parameter values (e.g., without clinician intervention) or may otherwise provide a prompt or recommendation to select or adjust one or more electrical stimulation parameter values (e.g., such as via programmer 20). In some examples, a sensed level of endothelization is utilized along with other factors (e.g., symptoms, side effects, or other sensed parameters) to select or adjust one or more stimulation parameters. Endothelization proximate endovascular device 16 and/or plurality of electrodes 17 may facilitate the ability to provide stimulation therapy or sense patient parameters, which may enhance the fidelity of therapy. In some examples, when a sufficient level of endothelization has been reached, a need for anticoagulation therapy (e.g., commonly dual antiplatelet therapy (DAPT)) is reduced or eliminated. For example, anticoagulation therapy may be provided to patient 12 to reduce formation of a thrombi on exposed surfaces of the plurality of electrodes 17. In response to receiving an indication that the sufficient level of endothelialization has been reached, a clinician may reduce or discontinue the anticoagulation therapy, thereby increasing an innate healing function of patient 12.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals may be processed by an analog-to-digital converter of processing circuitry 30 or other component to convert the conditioned analog signals into digital signals. In some examples, processing circuitry 30 may operate on the analog or digital form of the signals to separate out different components of the signals. In some examples, sensing circuitry 36 and/or processing circuitry 30 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

Although shown as part of medical device 14 in FIG. 2, in other examples, sensing circuitry 36 can be a part of a device separate from medical device 14. For example, sensing circuitry 36 can be part of an implantable sensing device implanted in cranial vasculature or elsewhere in brain 18 of patient 12.

Processing circuitry 30, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, control circuitry may include multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20, may accomplish communication by any suitable communication techniques, such as RF communication techniques. In addition, telemetry circuitry 38 may communicate with programmer 20 via proximal inductive interaction of medical device 14 with programmer 20. Accordingly, telemetry circuitry 38 may send information to programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

As discussed above, in some examples, endovascular device 16 is configured to be a standalone electrical stimulation device and can include one or more elements of medical device 14 shown in FIG. 2.

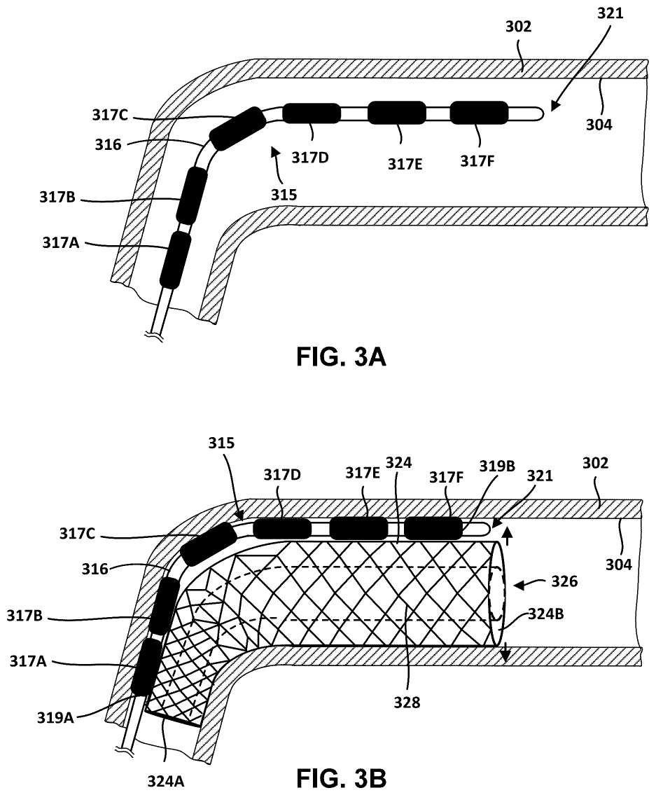

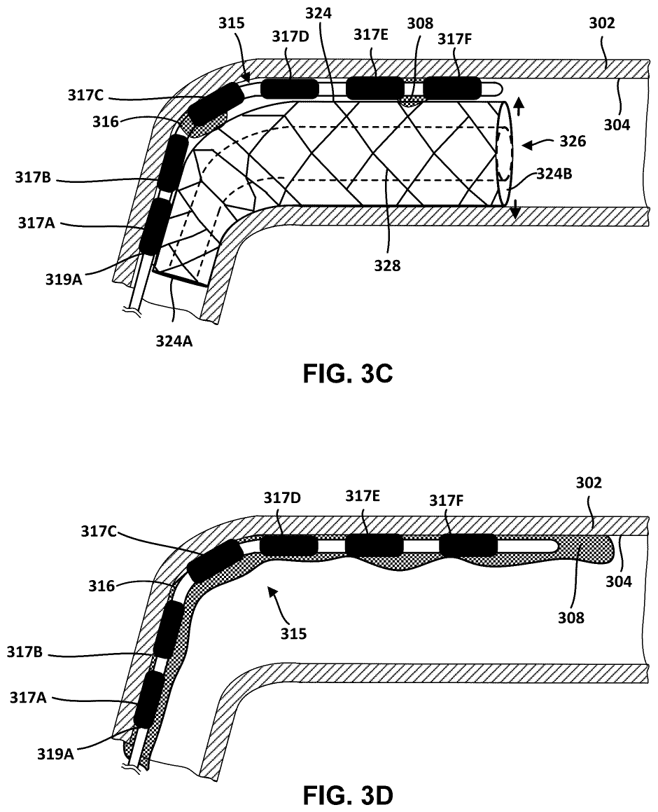

FIGS. 3A, 3B, 3C, and 3D illustrate an example distal portion 315 of an example endovascular device 316, which may be a medical lead, and a plurality of electrodes 317 (shown individually as electrode 317A, electrode 317B, electrode 317C, electrode 317D, electrode 317E, and electrode 317F, but collectively referred to herein as plurality of electrodes 317), as well as an elongated scaffold 328. Distal portion 315 of endovascular device 316 including plurality of electrodes 317 may be an example of distal portion 15 of endovascular device 16 including plurality of electrodes 17 described above, and elongated scaffold 328 may be an example of scaffold 28 described above.

As illustrated in FIGS. 3B and 3C, elongated scaffold 328 may include a tubular body 324 configured to an exert a radially outward (e.g., outward from a central longitudinal axis of tubular body 324) force to urge endovascular device 316 and/or plurality of electrodes 317 against a vessel wall 304 of a blood vessel 302. Tubular body 324 extends between a proximal face 324A (which also may be referred to as elongated scaffold proximal end 324A) and a distal face 324B (which also may be referred to as elongated scaffold distal end 324B), and defines an outer surface configured to contact portions of endovascular device 316, plurality of electrodes 317, and/or the inner wall 304 of vessel 302. In some examples, scaffold 328 is configured to exert a uniform radial force along the length (e.g., the axial length of scaffold 328), such that the force at all points around and along scaffold 328 exert a uniform outward force. However, in some examples, scaffold 328 is configured to target certain areas with more or less force, such that the radial force exerted by scaffold 328 is not uniform. For example, scaffold 328 may be configured to exert a greater force at one or more longitudinal locations along scaffold 328, e.g., to correspond to the longitudinal locations along scaffold 328 where scaffold 328 contacts each electrode of the plurality of electrodes 317. As another example, scaffold may be configured to exert a greater force at one or more radial locations around scaffold 328, e.g., to correspond to the radial location where scaffold 328 contacts each electrode of plurality of electrodes 317. In some examples, scaffold 328 is configured to exert a radial force to urge endovascular device 316 and/or plurality of electrodes 317 toward a particular part of the vessel wall (e.g., an inner or outer part of a curve of a vessel). For example, scaffold 328 may be configured such that electrodes 317 face outward (e.g., radially outward) in a particular direction to face toward nerves or other tissue outside of the blood vessel.

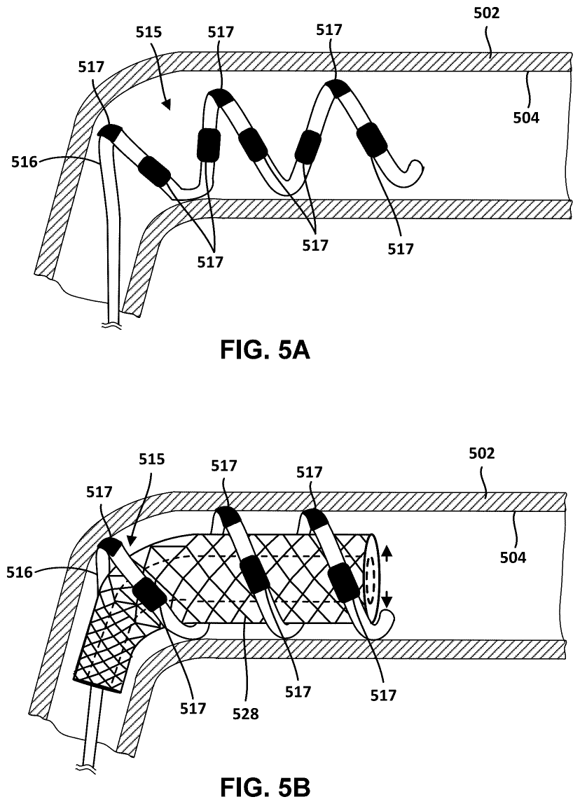

As illustrated in FIGS. 3A-3D, endovascular device 316 includes an elongated body with electrodes 317 disposed on, carried by, or otherwise defined by the elongated body of endovascular device 316. As discussed above, electrodes 317 may be configured to deliver electrical stimulation therapy to tissue (e.g., brain tissue or other nerve tissue surrounding blood vessel 302), or sense a patient parameter (e.g., signal) from a location within blood vessel 302. Plurality of electrodes 317 includes multiple, discrete electrodes axially spaced apart along distal portion 315 of endovascular device 316. However, endovascular device 316 may include any suitable number of electrodes with any suitable axial spacing (e.g., one electrode, two electrodes, three electrodes, four or more electrodes, etc.). The number and spacing of electrodes 317 may correspond to the therapy being delivered (and/or sensing configuration, in examples where electrodes 317 are configured to sense a patient parameter), the implant location (e.g., the specific blood vessel), or to account for specific patient factors (e.g., biological indicator such as age or gender, a disease state, a blood pressure, and/or a blood velocity). In some examples, when implanted into blood vessel 302, a portion of the elongated body of endovascular device 316 (e.g., distal portion 315) positioned adjacent elongated scaffold 328 is configured to linearly extend between a proximal end of the elongated scaffold and a distal end of the scaffold (e.g., such that endovascular device does not wrap circumferentially around elongated scaffold 328). However, in other examples, a portion of the elongated body of endovascular device 316 (e.g., distal portion 315) positioned adjacent elongated scaffold 328 is configured to coil around elongated scaffold 328 between a proximal end of the elongated scaffold and a distal end of the scaffold (as shown and discussed with respect to FIGS. 5A and 5B).

In some examples, a length (e.g., an axial length) of tubular body 324 of scaffold 328 corresponds to the position, orientation, and size of endovascular device 316 and/or plurality of electrodes 317. In some examples, when implanted, a distal end (e.g., distal face 324B, which may be a distal-most end) of scaffold 328 extends distally of distal-most electrode 317F and/or a distal end 319A of the plurality of electrodes 317. In some examples, when implanted, the distal end (e.g., distal face 324B) of scaffold 328 extends distally of a distal end 321 (which may be a distal-most end 321) of endovascular device 316. However, in some examples, when implanted, distal end 321 of endovascular device 316 and/or distal end 319B of plurality of electrodes 317 extends distally of a distal end (e.g., distal face 324B) of scaffold 328. In some examples, when implanted, a proximal end (e.g., proximal face 324A, which may be a proximal-most end) of scaffold 328 extends proximally of a proximal-most electrode 317A and/or proximal end 319A of plurality of electrodes 317. However, in some examples, when implanted, proximal end 319B of plurality of electrodes 317 extends proximally of the proximal end (e.g., proximal face 324A) of scaffold 328.

In some examples, scaffold 328 includes one or more sub-structures or components that enable scaffold 328 to apply a radially outward force (e.g., even when scaffold 328 begins to degrade). In some examples, scaffold 328 may be configured to retain a shape (e.g., a circular shape), even when scaffold 328 begins to degrade or resorb, as some sub-structures may be configured to remain intact while other portions of scaffold 328 begin to degrade or resorb. Scaffold 328 of may include a plurality of struts, coils, or other braid-like structures. Scaffold 328 may include any suitable braid pattern (e.g., 1 by 1, 2 by 1, 3 by 1, etc.) with any suitable pick count. In some examples, the struts, coils, or other braid-like structures are uniformly shaped and uniformly spaced along the length (e.g., the axial length) of scaffold 328. In other examples, as illustrated later, the struts, coils, or other braid-like structures of scaffold 328 are not uniformly shaped and/or are not uniformly spaced. In some examples, scaffold 328 includes at least one of a braided stent, laser-cut tube, or a formed wire mesh. In some examples, scaffold is formed via an injection molding process (e.g., in the case of thermoplastic materials) and/or through a deposition procession (e.g., in the case of a thermoplastic or Magnesium alloy materials).

Scaffold 328 may be configured to conform to the inner diameter of a blood vessel (e.g., as defined by blood vessel wall 304), such as to exert a uniform radial force along the length (e.g., the axial length) of scaffold 328 and/or exert a uniform radial force around the perimeter of scaffold 328. In some examples, as illustrated in FIG. 3B and 3C, scaffold 328 is configured to be placed around a curve of blood vessel 302. In some examples, tubular body 324 of scaffold 328 includes a pre-shaped curve configured to conform to a curve in vessel 302. In some examples, tubular body 324 of scaffold 328 is configured to conform around endovascular device 316 while maintaining contact with vessel wall 304 of blood vessel 302. For example, tubular body 324 may be configured to conform to or deform in a manner proximate to endovascular device 316, such that portions of tubular body 324 proximate to endovascular device 316 may provide a platform on which endothelial cells may propagate and migrate.

In some examples, tubular body 324 of elongated scaffold 328 defines an inner lumen 326, which may extend between a proximal face 324A of the tubular body 324 and a distal face 324B of the tubular body 324. Lumen 326 may be configured to allow blood to flow in and through tubular body 324 of elongated scaffold 328. In some examples, inner lumen 326 is sized to reduce the occlusive nature of scaffold 328, such as to reduce occlusion of blood flow through blood vessel 302. Lumen 326 may define a constant diameter, however the diameter of lumen 326 when scaffold 328 is implanted in vessel 302 may change, e.g., in response to the tubular body 324 of scaffold 328 conforming to vessel 302.

In some examples, such as illustrated in the example of FIG. 3A, endovascular device 316 including plurality of electrodes 317 may be delivered alone (e.g., delivered separately) and independent from (e.g., before) scaffold 328. However, in some examples, endovascular device 316 including plurality of electrodes 317 and elongated scaffold 328 are configured to be delivered concurrently (e.g., delivered together via the same delivery tool, including a delivery catheter or another delivery device) to a target location within the neurovasculature of patient 12. In examples where endovascular device 316 including plurality of electrodes 317 are delivered concurrently with elongated scaffold 328, endovascular device 16 may be secured (e.g., coupled to) elongated scaffold 328 using any suitable mechanism. For example, endovascular device 316 and/or plurality of electrodes 317 may be bonded to, interwoven into (e.g., FIG. 10), carried by, or otherwise coupled to scaffold 328.

As shown in the progression of FIGS. 3B, 3C, and 3D, scaffold 328 may be configured to reabsorb or degrade (e.g., via hydrolysis, such as from water in the blood) while plurality of electrodes 317 and/or portions of endovascular device 316 become incorporated into blood vessel 302 via endothelization. For example, the example of FIG. 3B may correspond to a first timepoint after scaffold 328 and endovascular device 316 with plurality of electrodes 317 are initially implanted, e.g., when no or minimal endothelization has occurred. The example of FIG. 3C may correspond to a second timepoint after the first timepoint when scaffold 328 has started to reabsorb or degrade, and some endothelization has taken place, indicated by formation of tissue 308 around some of plurality of electrodes 317 and endovascular device 316. The example of FIG. 3D may correspond to a third timepoint after the first and the second timepoints in which scaffold 328 has completely reabsorbed or degraded, and plurality of electrodes 317 has undergone more endothelization, indicated by tissue 308 completely covering electrodes 317 and a portion of endovascular device 316.

In some examples, the degradation profile of scaffold 328 is based on one or more of a thickness of scaffold 328 (e.g., a thickness of a wall of the tubular body 324 of scaffold 28 and/or a thickness of individual struts of scaffold 328), a material of scaffold 328, an arrangement of materials within scaffold 328, or the like. As described below, scaffold 328 can include one or more materials that degrade and/or resorb into the vasculature. The arrangement of materials (e.g., via layering) may affect the degradation profile. A sizing and arrangement of struts of other sub-structures of scaffold 328 may affect the degradation profile. For example, increasing thickness of struts of scaffold 328 may cause scaffold 328 to degrade more slowly.

Scaffold 328 may include any suitable material or combination of materials configured to absorb or degrade after implanted in the blood vessel of a patient. In some examples, scaffold 328 includes a biodegradable and/or bioresorbable polymer. In some examples, scaffold 328 includes one or more of Polyglycolic acid (PGA), Polylactic acid (PLA), Polylactic-coglycolic acid (PLGA), Polycaprolactone (PCL), Polydioxanone (PDO), Polyethylene glycol (PEG), or Polytrimethylene carbonate (PTMC). In some examples, scaffold 328 includes Magnesium or a Magnesium alloy. As discussed below, scaffold 328 (e.g., the struts, braids, or other portions of scaffold 328) may include a combination of materials such that scaffold 328 has a multi-phasic degradation profile, e.g., where scaffold 328 degrades more quickly or more slowly during a first phase as compared to a second phase. While the materials discussed in this disclosure are primarily configured to degrade via hydrolysis reaction, scaffold 28 may be configured to degrade via any suitable mechanism (corrosion, catalyzed reaction, etc.).

In some examples, scaffold 328 additionally includes one more radiopaque or radiographic materials, such as to visualize scaffold 328 under a suitable medical imaging technique such as fluoroscopy. The radiopaque or radiographic materials may enable visualization during placement of scaffold 328 within the vasculature. In some examples, one or more radiopaque markers may be affixed to scaffold 328. Radiopaque markers may include one or more of platinum, platinum-iridium, or other suitable metals or alloys. Radiopaque markers may be affixed to a distal and/or proximal end of scaffold 328, such as to visualize the proximal and/or distal end of scaffold. Visualization may be in relation to one or more anatomical features, or in relation to other device components, e.g., a proximal-most electrode 317A, a distal-most electrode 317F, a proximal end 319A and/or a distal end 319B of plurality of electrodes 317. In some example, one or more radiopaque markers may additionally be attached between the proximal and distal end of scaffold 328. Including multiple radiopaque markers may enable visualization of whether scaffold 328 is longitudinally extended or compressed. In some examples, the struts and/or braids of scaffold 328 may be compounded with a radiopaque material. Other radiopaque materials, that may be used to affix markers to scaffold 328 or be compounded with the materials of scaffold 328 (e.g., the struts or braid portions of scaffold 328) include barium sulfate, tungsten, or other suitable materials. Endovascular device 316 may additionally or alternatively include any of the aforementioned radiopaque markers and/or materials.

While the examples of FIGS. 3B and 3C illustrate a single scaffold 328 being used to urge endovascular device 316 and/or plurality of electrodes 317 against vessel wall 304, any suitable number of scaffolds may be used. For example, multiple scaffolds may be implanted blood vessel 302 and longitudinally spaced apart to apply a force to different regions of endovascular device 316. For example, a second scaffold may be placed adjacent a more proximal portion of endovascular device 316, e.g., proximal to distal portion 315 and/or proximal to plurality of electrodes 317. In some examples, multiple scaffolds are placed to apply a radially outward force on particular portions of endovascular device 316, e.g., only on portions where plurality of electrodes 317 are located along the length of endovascular device 316.

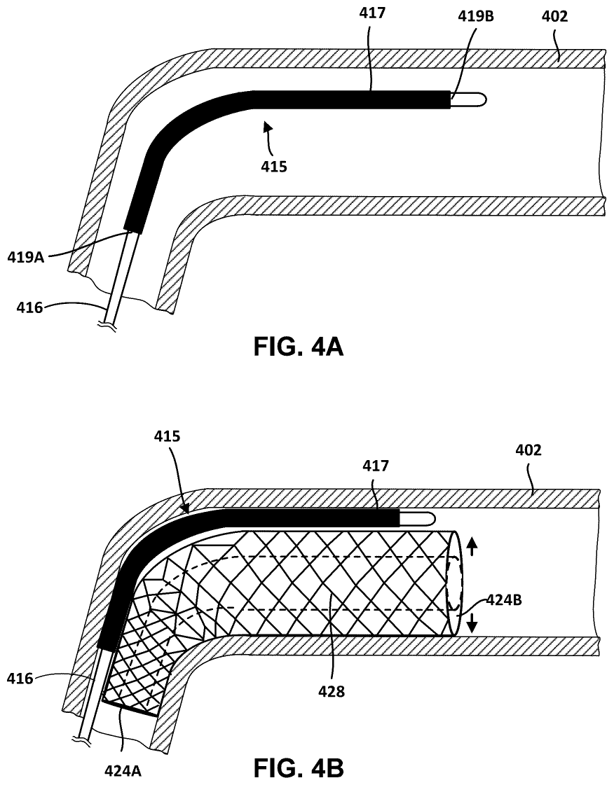

While the example scaffold 328 is illustrated as urging an endovascular device 316 that includes multiple electrode 317 against vessel wall 304, example scaffolds described herein may be used to urge any number or configuration of electrodes against vessel wall 304. FIGS. 4A and 4B illustrate an example distal portion 415 of an example endovascular device 416, which may be a medical lead, with an electrode 417 and an elongated scaffold 428 within a vessel 402. Distal portion 415 of endovascular device 416 including electrode 417 may be an example of distal portion 15 of endovascular device 16 including plurality of electrodes 17 described above, and elongated scaffold 428 may be an example of scaffold 28 described above. Endovascular device 416, electrode 417, and scaffold 428 otherwise include all of the attributes of endovascular device 316, plurality of electrodes 317, and scaffold 328 described in connection with FIGS. 3A-3D, except as described herein.

In the example of FIG. 4A and FIG. 4B, endovascular device 416 includes a single elongated electrode 417 rather than multiple discrete and axially spaced-apart electrodes, as discussed in connection with FIGS. 3A-3D. A single elongated electrode 417 may enable stimulation over a longer axial distance (e.g., along a blood vessel), such as compared to shorter electrodes. The single elongated electrode 417 may enable tuning of surface area for choosing charge injection capacity. In some examples, electrode 417 defines a length that corresponds to an axial length of scaffold 428. In some examples, electrode 417 defines an axial length that is about 70 percent, 80, percent, 90 percent, or more of the axial length of scaffold 428. The axial length of electrode 417 may be the axial length between a proximal-most end 419A of electrode 417 and a distal-most end 419B of electrode 417 when electrode 417 is straight. The axial length of scaffold 428 may be the axial length between a proximal-most end 424A of scaffold 428 and a distal-most end 424B of scaffold 428.

FIGS. 5A and 5B illustrate an example distal portion 515 of an example endovascular device 516, which may be a medical lead, with a plurality of electrodes 517, as well as an elongated scaffold 528. Distal portion 515 of endovascular device 516 including electrodes 517 may be an example of distal portion 15 of endovascular device 16 including plurality of electrodes 17 described above, and elongated scaffold 528 may be an example of scaffold 28 described above. Endovascular device 516, electrodes 517, and scaffold 528 otherwise include all of the attributes of endovascular device 316, plurality of electrodes 317, and scaffold 328 described in connection with FIGS. 3A-3D, except as described herein.

In the example of FIG. 5A and 5B, endovascular device 516 includes an elongated body, where a portion of elongated body of endovascular device 516 is configured to wrap or coil around elongated scaffold 528. For example, as illustrated in FIG. 5B, distal portion 515 of endovascular device 516, which includes electrodes 517 and is positioned adjacent to scaffold 528, defines a coil shape. The coil shape may be a pre-formed coil shape. The pre-formed coil portion of endovascular device 516 may be configured to self-expand. In this way, endovascular device 516, including the elongated body, may be configured to position one or more electrodes of electrodes 517 in apposition with (e.g., in contact with) the blood vessel wall 504 of blood vessel 502. The pre-formed coil shape may enable endovascular device 516 to hold electrodes 517 in opposition with vessel wall 504 without scaffold 528. However, once positioned within the coil shape of endovascular device 516, scaffold 528 may be configured to further urge electrodes 517 and/or a portion of endovascular device 516 against vessel wall 504.

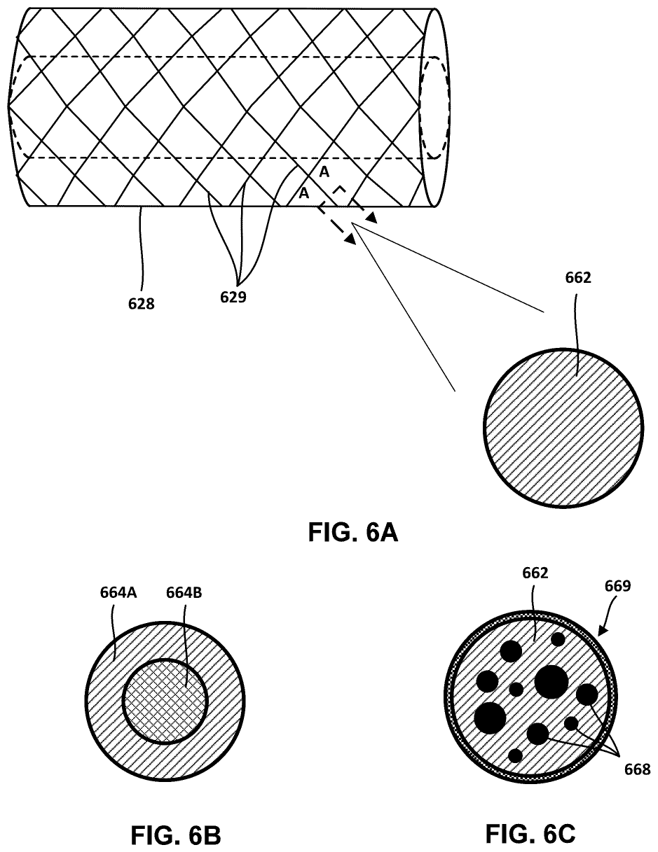

FIGS. 6A, 6B, and 6C illustrate various examples of cross-sections of struts and/or braids of elongated scaffolds according to the techniques of this disclosure. FIG. 6A illustrates an example elongated scaffold 628 with a plurality of struts 629, and a cross-sectional view of a single strut (indicated as the “A-A” cross-section). FIGS. 6B and 6C also illustrate examples of cross-sectional views of a single strut of struts 629 taken along the “A-A” cross section of FIG. 6A. Scaffold 628 may be an example of any of the elongated scaffolds discussed previously (e.g., scaffold 28, scaffold 328, scaffold 428, or scaffold 528). While the cross-sections of FIGS. 6A, 6B, and 6C illustrate plurality of struts 629 having circular cross section, struts 629 may have any desired cross-sectional shape or combination of cross-sectional shapes (e.g., circular, semi-circular, ovular, square, rectangular, or other suitable shapes).

The plurality of struts 629 of elongated scaffold 628 may include a material or combination of materials in any suitable microscopic or macroscopic arrangement, e.g., to effectuate a desired degradation profile (e.g., reduction of material over time, degradation rate, etc.) of scaffold 628. In some examples, scaffold 628 can include any suitable arrangement of strut sections (e.g., connected by bridges) and/or reinforced strut sections, e.g., to effectuate a desired degradation profile and/or radial force profile. In the example of FIG. 6A, plurality of struts 629 includes a core 662, which may include a homogenous (e.g., uniform) material throughout core 662. Core 662 occupies the entire cross section of struts 629. In the example of FIG. 6B, plurality of struts 629 includes an outer core 664A and an inner core 664B, where inner core 664B is radially inward from and at least partially surrounded by outer core 664A. In some examples, outer core 664A includes a first material (also referred to herein as first core material) and inner core 664B includes a second material (also referred to herein as second core material). In this way, each strut of struts 629 includes a first material (e.g., outer core 664A) that at least partially surrounds the second material (e.g., inner core 664B). In some examples, the first material of outer core 664A is different than the second material of inner core 664B. In some examples, the first material of outer core 664A is the same material as second material of inner core 664B, but has a different molecular weight (e.g., in examples including polymeric materials where molecular weight can be adjusted). In some examples, the second material of inner core 664B is configured to resorb or degrade faster than the first material of outer core 664A. Said another way, the first material of outer core 664A may be configured to resorb or degrade more slowly than the second material of inner core 664B. By using two or more different materials for the plurality of struts 629, scaffold 628 may be configured to apply a radial force (e.g., on elongated body of an endovascular device and/or electrodes of an endovascular device, as described above) for a first period of time while the outer core 664A resorbs or degrades, and subsequently rapidly degrade during a second period of time when the outer core 664A has degraded leaving only the faster degrading second material of inner core 664B.

As illustrated in FIG. 6C, struts 629 may one or more of a coating 669 or a filler material 668. Coating 669 may be an external coating applied to the outer surface of struts 629. In some examples, coating 669 may is uniformly applied around each strut (e.g., as shown in FIG. 6C), such that coating 669 encapsulates the entire outer surface of struts 629. Filler material 668 may include particles or a compounded material that is otherwise incorporated into the material matrix of a struts 629.

In some examples, scaffold 628 is configured to release (e.g., elute) a therapy agent into a blood vessel when implanted in a patient (e.g., an anti-thrombogenic agent to reduce thrombosis, an antiplatelet agent, an anticoagulant agent, or another suitable agent configured to cause a therapeutical outcome). In some examples, coating 669 (which may be an external coating) includes the therapy agent. In some examples, when scaffold 628 including outer coating 669 is delivered into a blood vessel, the outer coating 669 may provide an initial release of the therapy agent into the bloodstream before scaffold 628 begins to resorb or degrade. In some examples, filler material 668 additionally or alternatively includes the therapy agent. By including filler material 668 within struts 629, scaffold 628 may be configured to release the filler material (which may include the therapy agent) over time, such as while each strut of struts 629 resorbs or degrades.

The example struts of examples of FIGS. 6A, 6B, and 6C, including the multi-core configuration of FIG. 6B, and the coating and filler material configuration of FIG. 6C may be used in any combination or permutation. For example, the multi-core configuration of FIG. 6B may include coating 669 on an outer surface of outer core 664A. As another example, either or both of outer core 664A or inner core 664B may include filler material 668.

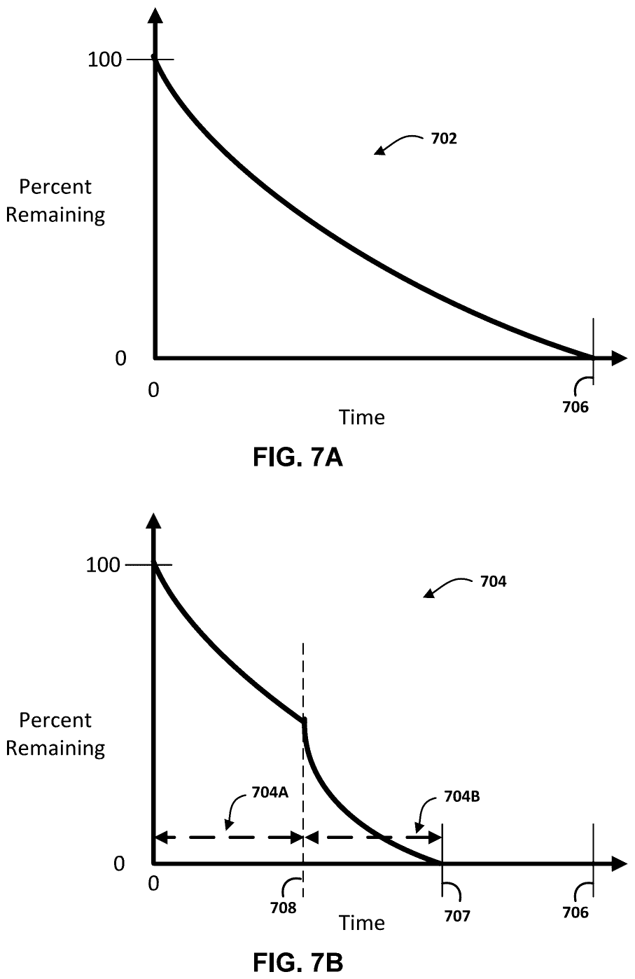

FIGS. 7A and 7B include graphs illustrating example degradation profiles of scaffolds according to the techniques of this disclosure. FIG. 7A includes a graph 702, which may be an example degradation profile of an example scaffold according to the techniques of this disclosure. For example, graph 702 may be a degradation profile of an elongated scaffold including a single core material, as illustrated in connection with core 662 of FIG. 6A. FIG. 7B includes a graph 704, which may be an example degradation profile of an example scaffold according to the techniques of this disclosure including at least two different core materials. For example, graph 704 may be a degradation profile of an elongated scaffold including at least two different core materials, as illustrated in connection with core 664A and inner core 664B of FIG. 6B.

Graph 702 and graph 704 illustrate example degradation profiles of elongated scaffolds, with the y-axis of each of graph 702 and graph 704 including a “percent remaining” of the elongated scaffold and the x-axis including “time” (e.g., which may include any suitable measure of time, such as second, minutes, hours, days, months, etc.). The “percent remaining” of the elongated scaffold may include any suitable measure of the reduction of a scaffold (e.g., mass, volume, etc.). The y-axis ranges from 100 percent (e.g., no degradation) to 0 percent (complete degradation). For graph 702, the x-axis ranges from 0 to timepoint 706, where timepoint 706 includes the time at which a scaffold is completely degraded within the vasculature. For graph 704, the x-axis also ranges from 0 to timepoint 707 where timepoint 707 includes the time at which a scaffold is completely degraded within the vasculature, but also includes timepoint 708 between 0 and timepoint 707. Timepoint 708 may indicate a timepoint at which a first core material has completely absorbed or degraded to expose a second core material, and the second core material has begun to degrade.

In the example of FIG. 7A, graph 702 illustrates an example of a degradation rate of a single core scaffold during a time period from 0 to timepoint 706. Graph 702 may be scaled to account for changes in surface area during degradation or resorption, and is merely illustrative of a single core scaffold in which a single material degrades or resorbs over time. In some examples, the degradation rate, even of a single material scaffold, may change more or less over time due to changes in surface area (e.g., surface area of struts exposed to the vasculature), changes in blood flow, or other physical factors. While graph 702 illustrates a an example degradation rate, a single core scaffold may in reality (e.g., during in vivo implantation) have a degradation rate that increases more or decreases more over time as compared to what is shown in graph 702. For example, a single core scaffold may initially have a faster rate of degradation, and subsequently taper to a slower rate of degradation as time progresses.

In the example of FIG. 7B, graph 704 illustrates an example of a non-uniform degradation rate from time 0 to timepoint 707 (e.g., two different rates of degradation or resorption corresponding to a dual core scaffold). Graph 704 may be scaled to account for changes in surface area during degradation or resorption, and is merely illustrative of a dual core scaffold in which two core materials degrade or resorb over time. Graph 704 illustrates a first time period 704A including a first degradation rate, which may correspond to a first core material (e.g., as discussed in connection with outer core 664A of FIG. 6B). Graph 704 also illustrates a second time period 704B including a second degradation rate, which may correspond to a second core material (e.g., as discussed in connection with inner core 664B of FIG. 6B). In the example of graph 704, the second degradation rate beginning at timepoint 708 and ending at timepoint 707 and associated with the second time period 704B is faster than the first degradation rate associated with the first time period 704A, as indicated by the greater slope of the line representing the percent remaining versus time in the second time period 704B as compared to the first time period 704A. As shown in graph 704, the dual core scaffold may completely degrade before timepoint 706, illustrating that the dual core scaffold may degrade faster than a single core scaffold as illustrated in graph 702 of FIG. 7A.

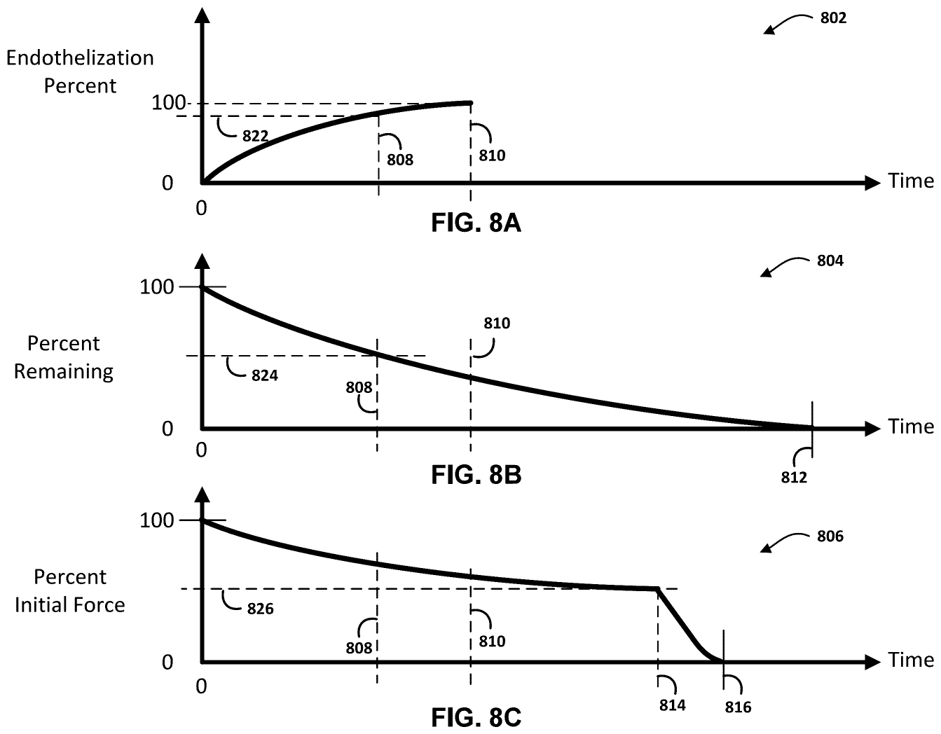

FIGS. 8A, 8B, and 8C include graphs illustrating an example rate of endothelization (e.g., of an endovascular device and/or a plurality of electrodes), as compared to an example rate of degradation of an elongated scaffold, as compared to the decline in radial force provided by an elongated scaffold according to the techniques of this disclosure. FIG. 8A includes a graph 802, which may be an example endothelization profile (e.g., endothelization versus time) of an endovascular device and/or a plurality of electrodes (e.g., such as any of endovascular device 16, endovascular device 316, endovascular device 416, endovascular device 516 and/or electrodes 17, electrodes 317, electrode 417, electrodes 517 as discussed previously). FIG. 8B includes a graph 804, which may be an example degradation profile of an example scaffold (e.g., any of elongated scaffold 28, elongated scaffold 328, elongated scaffold 428, elongated scaffold 528, elongated scaffold 628, etc.) according to the techniques of this disclosure (e.g., as discussed in connection with graph 702 and graph 704 of FIGS. 7A and 7B). For example, graph 804 may be a degradation profile of an elongated scaffold including a single core material, as illustrated in connection with core 662 of FIG. 6A. FIG. 8C includes a graph 806, which may be an example profile of radial force versus time, wherein the radial force is provided by an example elongated scaffold (e.g., any of elongated scaffold 28, elongated scaffold 328, elongated scaffold 428, elongated scaffold 528, elongated scaffold 628, etc.). Graph 802, graph 804, and graph 806 are presented on concurrent timelines such as to illustrate the interplay of endothelization of the endovascular devices and/or electrodes and the degradation and force profiles of the elongated scaffolds according the to the techniques of this disclosure. While graph 802, graph 804, and graph 806 may apply to any of the elongated scaffolds, endovascular devices, and electrodes described in this disclosure, the graphs will be described with respect to elongated scaffold 28, endovascular device 16, and plurality of electrodes 17 as shown and described in connection with FIG. 1. In some examples, one or more of a material selection, scaffold design (e.g., strut count, strut diameter), or other factors (e.g., patient-specific factors) may affect how graphs 802, graph 804, and graph 806 look in reality, and these graphs are merely illustrative examples for example elongated scaffolds, endovascular devices, and electrodes.

Graph 802 of FIG. 8A illustrates an example endothelization profile (e.g., endothelization percentage versus time as indicated by a line) of endovascular device 16 and/or plurality of electrodes 17, with the y-axis of graph 802 including a scale of “endothelization percent,” which ranges from 0 percent to 100 percent, wherein 0 percent indicates no endothelization and 100 percent indicates complete endothelization (e.g., a point at endovascular device 16 and/or plurality of electrodes 17 ceases becoming more incorporated into a vessel wall). The x-axis of graph 802 includes “time” (e.g., which may include any suitable measure of time, such as second, minutes, hours, days, months, etc.), and ranges from 0 to timepoint 810, wherein timepoint 810 correspond to 100 percent endothelization. In some examples, timepoint 810 occurs at about 10 to 30 days, such as at about 20 days. In some examples, timepoint 810 occurs at about 50 to 70 days, such as about 60 days. In some examples, timepoint 810 occurs around 10 to 70 days, such as about 40 days. However, timepoint 810 may occur at a shorter or longer time than the aforementioned examples. The y-axis of graph 802 also indicates a threshold endothelization level 822, which corresponds to a timepoint 808. Threshold endothelization level 822 may include a level at which a sufficient level of endothelization of endovascular device 16 and/or electrodes 17 has occurred, which may be characterized by the electrodes 17 exhibiting a threshold level of impedance, or characterized by another measurable parameter. In some examples, timepoint 808 occurs at about 10 to 30 days, such as at about 20 days (but may occur at in a small or longer period of time). In some examples, timepoint 808 is sooner or slightly sooner than timepoint 810 (e.g., such as 1 day, 2 days, 3 days, or any suitable length of time). In some examples, timepoint 808 and timepoint 810 are the same (e.g., where the threshold level of endothelization is 100 percent endothelization). As impedance of electrodes 17 is reduced as endothelization progresses (e.g., as endovascular device 16 and/or electrodes 17 become incorporated into a vessel wall), impedance may serve as a proxy to measure endothelization. Therefore, 100 percent endothelization may include an impedance level that is stable (e.g., no longer changing). In some examples, threshold endothelization level 822 includes a level of endothelization in which the radial force of elongated scaffold 28 is no longer needed to urge endovascular device 16 and/or electrodes 17 against a vessel wall.

Graph 804 of FIG. 8B illustrates an example degradation profile of elongated scaffold 28, with the y-axis of graph 804 including a “percent remaining” of elongated scaffold 28 and the x-axis including “time” (e.g., which may include any suitable measure of time, such as second, minutes, hours, days, months, etc.). The y-axis ranges from 100 percent (e.g., no degradation) to 0 percent (complete degradation), and may include any suitable measure of the reduction of a scaffold (e.g., by mass, volume, etc.). For graph 804, the x-axis ranges from 0 to timepoint 812, where timepoint 812 corresponds to a time at which scaffold 28 is completely degraded within the vasculature. Graph 804 of FIG. 8B may be an example of the degradation profile of graph 702 as described in connection with FIG. 7A. At timepoint 808 (which corresponds with threshold endothelization level 822 of FIG. 8A), graph 804 illustrates a corresponding percent remaining level 824 of scaffold 28. In some examples, percent remaining level 824 is about 40 percent to 60 percent, such as about 50 percent. As can be seen by comparing FIGS. 8A and 8B, timepoint 812 (in which scaffold 28 is completely degraded) occurs after both timepoint 808 (which corresponds with threshold endothelization level 822 of FIG. 8A) and timepoint 810 (which corresponds to 100 percent endothelization level of FIG. 8A). However, in some examples, timepoint 812 (in which scaffold 28 is completely degraded) occurs after only timepoint 808, but before timepoint 810. In other words, scaffold 28 may only remain intact (and able to provide a radial force, as discussed below), until a threshold (e.g., sufficient) level of endothelization has occurred. As an illustrative example, if electrodes 17 take 20 days to incorporate (e.g., either reach timepoint 808 and/or timepoint 810), scaffold 28 may exhibit 50 percent degradation by 30 days and 95 percent degradation by 60 days from time “0.”

As illustrated in graph 802 of FIG. 8A and graph 804 of FIG. 8B, scaffold 28 is configured such that the degradation profile of elongated scaffold 28 over one or more periods of time (e.g., the time period until timepoint 808) corresponds to a level (e.g., threshold level 822) of endothelization proximate the plurality of electrodes. For example, scaffold 28 is configured such that the corresponding percent remaining level 824 of scaffold 28 at timepoint 808 corresponds to scaffold 28 being able to provide a sufficient radial force to maintain contact of electrodes 17 against the wall of a blood vessel. Elongated scaffold 28 is configured to maintain contact of the plurality of electrodes with the wall of the blood vessel for at least a portion of the period of time until timepoint 812 (e.g., until timepoint 808, which occurs before timepoint 812). The portion of the period of time where the elongated scaffold maintains contact of the plurality of electrodes with the wall of the blood vessel (e.g., until timepoint 808) corresponds to threshold level of endothelization 822 proximate the plurality of electrodes.

Graph 806 of FIG. 8C illustrates an example force profile of elongated scaffold 28 over time, with the y-axis of graph 806 including “percent initial force” of elongated scaffold 28 and the x-axis including “time”, which may correspond to the same time of graphs 802 and 804. The y-axis ranges from 100 percent (e.g., the initial radial force provided by elongated scaffold 28 when initially implanted into the vasculature of a patient adjacent endovascular device 16 and/or plurality of electrodes 17). For graph 806, the x-axis ranges from 0 to timepoint 816, where timepoint 816 corresponds to a time in which scaffold 28 no longer provides any force (or provides a very small force) against endovascular device 16 and/or plurality of electrodes 17. For example, timepoint 816 corresponds to a point at which scaffold 28 has degraded enough to no longer contact endovascular device 16 and/or plurality of electrodes 17. However, timepoint 816 may still be before timepoint 812 of graph 804 (e.g., which corresponds to a time at which scaffold 28 is completely degraded), such that at least a portion of elongated scaffold 28 still remains in the vasculature even though it is not contacting or not providing a force against endovascular device 16 and/or plurality of electrodes 17. The radial force provided by scaffold may change over time, e.g., as a function of the strut diameter (in examples with circular or nearly circular strut cross sections), and/or as a function of strut width and/or thickness (in examples with rectangular or elliptical cross sections). For example, for circular cross sections, force may be proportional to strut diameter to the fourth power. As another example, force elliptical or rectangular cross sections, force may be proportional to width multiplied by thickness to the third power.

At timepoint 808 (which corresponds with threshold endothelization level 822 of FIG. 8A), graph 806 of FIG. 8C illustrates that the force provided by scaffold 28 (measured as a percent of initial force) is still above a threshold force 826, wherein threshold force 826 corresponds to a minimum radial force needed to effectuate apposition of endovascular device 16 and/or electrodes 17 against a vessel wall for endothelization. At a timepoint 814, which is after timepoint 808, graph 806 illustrates a point at which the radial force (measured as a percent of initial force) falls below threshold force 826. Thus graph 806 illustrates that scaffold 28 provides at least threshold force 826 at least until a threshold endothelization level 822 of endovascular device 16 and/or plurality of electrodes 17 has occurred. Thus, not only does scaffold 28 remain intact until after at least until a threshold endothelization level 822 of endovascular device 16 and/or plurality of electrodes 17 has occurred, but also provides threshold force 826 at least until a threshold endothelization level 822 of endovascular device 16 and/or plurality of electrodes 17 has occurred. In this way, elongated scaffold 28 is configured to exert at least threshold force 826 on plurality of electrodes 17 for the portion of the period of time to maintain contact of plurality of electrodes 17 with the wall of the blood vessel.

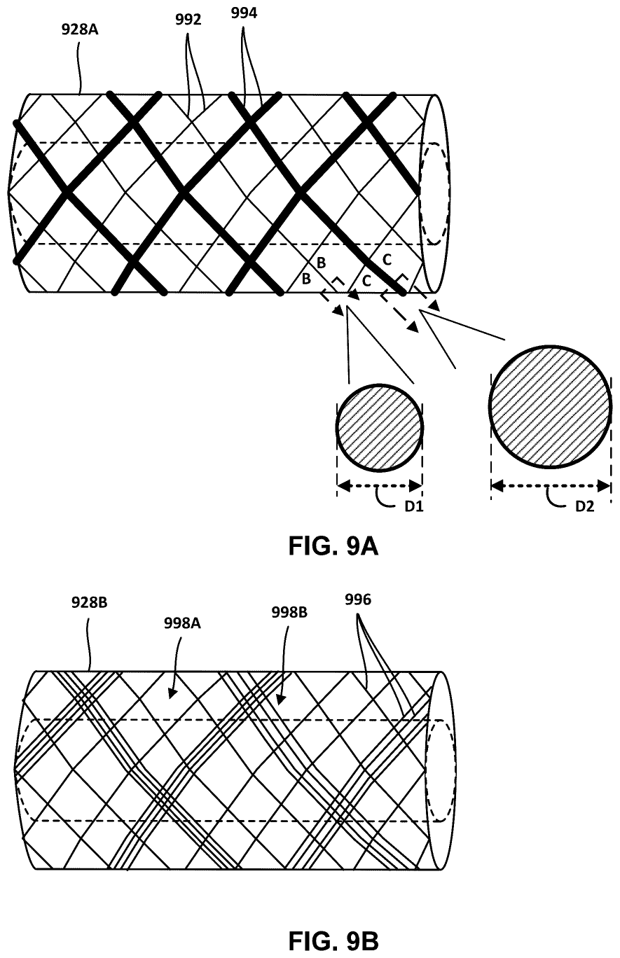

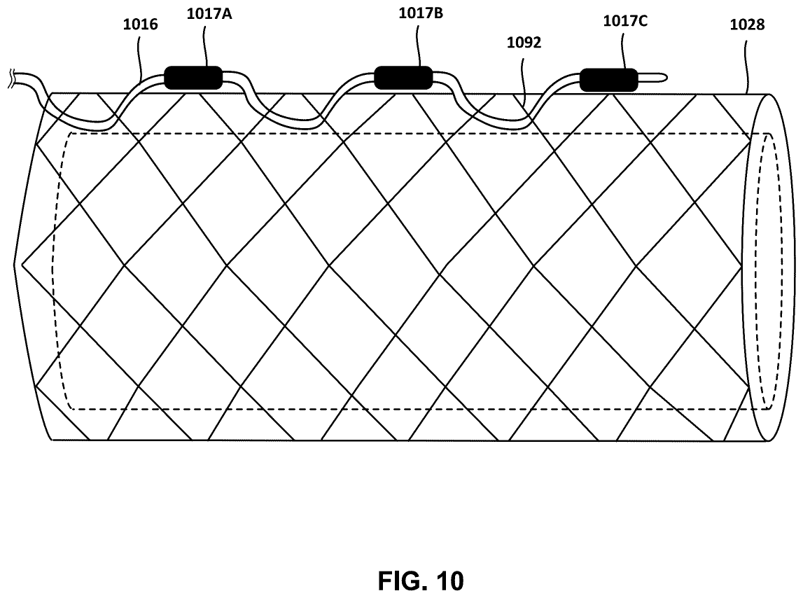

Various properties of scaffolds described herein, such as a degradation profile and an amount of force exerted, may be related to a size and arrangement of struts and/or braids. FIGS. 9A and 9B illustrate example strut and/or braid configurations of elongated scaffolds. FIG. 9A illustrates an example elongated scaffold 928A including varying strut size (e.g., thickness) and FIG. 9B illustrates an example elongated scaffold 928B with varying strut spacing, where each of scaffold 928A and scaffold 928B may be examples of the elongated scaffolds described previously (e.g., elongated scaffold 28, scaffold 328, scaffold 428, scaffold 528, or scaffold 628). Further, the example configurations of scaffold 928A and scaffold 928B described herein may be used in any combination or permutation with any of the scaffolds described previously.

In the example of FIG. 9A, scaffold 928A includes a first plurality of struts 992 having a first cross-sectional dimension D1 (e.g., as shown by a cross sectional view of a single strut of struts 992 taken along the “B-B” cross-section) and second plurality of struts 994 having a second cross-sectional dimension D2 (e.g., as shown by a cross sectional view of a single strut of struts 994 taken along the “C-C” cross-section). In examples in which struts 992 and struts 994 have circular cross-section, D1 and D2 may be diameters, however cross-sectional dimension D1 and/or cross-sectional dimensional D2 may be a greatest cross-sectional dimension in examples where struts 992 and/or struts 994 do not have circular cross sections. In some examples, cross-sectional dimension D2 of struts 994 is equal to cross-sectional dimension D1 of struts 992. However, in some examples, as illustrated in FIG. 9A, cross-sectional dimension D2 of struts 994 is greater than cross-sectional dimension D1 of struts 992. In some examples, cross-sectional dimension D2 is 10 percent greater than cross-sectional dimension D1, but may any size larger than D1 (e.g., 1 percent greater, 5 percent, 20 percent, 50 percent, 100 percent, 200 percent, etc.).

As illustrated in the example of FIG. 9A, scaffold 928A can include an alternating pattern of struts 992 and struts 994. For example, scaffold 928A can include an equal alternating pattern of first plurality of struts 992 and second plurality of struts 994. In some examples, scaffold 928A includes patterns to increase a strut density of first plurality of struts 992 as compared to second plurality of struts 994. For example, scaffold 928A may include more struts of first plurality of struts 992 as compared to struts of second plurality of struts 994. Alternatively, in some examples, scaffold 928A includes patterns to increase a strut density of second plurality of struts 994 as compared to the first plurality of struts 992. For example, scaffold 928A may include more struts of the second plurality of struts 994 as compared to struts of first plurality of struts 992. In some examples, scaffold 928A includes strut patterns where the density of either of first plurality of struts 992 and/or second plurality of struts 994 varies along an axial length of scaffold 928A.

Scaffold 928A may be configured to have portions (e.g., portions along an axial length of scaffold 928A or portions around the circumference of scaffold 928A) that provide a greater outer radial force or resist deformation more as compared to other portions of scaffold 928A. For example, portions of scaffold 928A having struts 994 with larger cross-sectional dimension D2 may be able to provide a greater outward radial force and/or resist deformation more as compared to portions of scaffold 928A having struts 992 having smaller cross-sectional dimension D1. In some examples, the portions configured to provide a greater outward radial force or resist deformation (e.g., portions with struts 994) may correspond to portions of scaffold 928A that contact electrodes of endovascular device (e.g., electrodes of 17, as described in connection with FIG. 1). In this way, scaffold 928A may be configured to be positioned adjacent an endovascular device 16 with electrodes 17 such that portions of scaffold 928A with struts 994 align (e.g., axially and/or circumferentially align) with where scaffold 928A contacts electrodes 17.

In the example of FIG. 9B, scaffold 928B includes a plurality of struts 996, where struts 996 including a first strut grouping 998A and a second strut grouping 998B. First strut grouping may include a grouping of struts 996 that have a first spacing between adjacent struts of struts 996. Second strut grouping 998B may include a group of struts 996 that have a second spacing between adjacent struts, where the second spacing is different that the first spacing. As illustrated, the struts of first strut grouping 998A may have a greater distance between adjacent struts as compared to struts of second strut grouping 998B.

FIG. 10 illustrates a side view of an example elongated scaffold 1028 including a plurality of struts 1092 with an endovascular device 1016 interwoven through elongated scaffold 1028 (e.g., an elongated body of endovascular device is woven through the struts 1092 of scaffold 1028). As illustrated in FIG. 10, endovascular device 1016 includes a plurality of electrodes 1017 (shown individually as electrode 1017A, electrode 1017B, and electrode 1017C, but collectively referred to herein as plurality of electrodes 1017) positioned along endovascular device 1016.

Even though portions of endovascular device 1016 are interwoven through the struts 1092 of scaffold 1028 (e.g., such that at least some portions of endovascular device 1016 are not positioned radially outside of scaffold 1028), scaffold 1028 may still be configured to urge electrodes 1017 against a blood vessel wall. For example, as illustrated in FIG. 10, electrodes 1017 may be positioned radially outside of scaffold 1028 such that when scaffold 1028 provides a radially outward force, electrodes 1017 are urged against a vessel wall. As discussed previously, urging electrodes 1017 against a blood vessel wall may promote endothelization, which may reduce the overall power needed to deliver efficacious electrical stimulation therapy to a patient, as well as help secure the electrodes in place in the blood vessel in instances of chronic therapy delivery.

As described above, interweaving endovascular device 1016 (or a portion thereof, including at least a portion of the elongated body of endovascular device 1016) within scaffold 1028 may enable scaffold 1028 and endovascular device 1016, including plurality of electrodes 1017, to be delivered contemporaneously (e.g., together, at the same time, via one delivery tool). Delivering scaffold 1028 and endovascular device 1016 together may enable delivery via a single catheter (e.g., delivery catheter). Delivering scaffold 1028 and endovascular device 1016 together may enable to be aligned (e.g., axially and radially aligned) before delivery, so as to avoid needing to reposition either of scaffold 1028 and/or endovascular device 1016 once implanted into the vasculature of a patient.

While the example of FIG. 10 shows endovascular device 1016 interwoven through scaffold 1028 that generally extending axially along scaffold 1028, endovascular device can be interwoven through scaffold 1028 in any suitable configuration. For example, endovascular device 1016 can be interwoven through scaffold 1028 such that endovascular device 1016 coils around scaffold 1028 (e.g., such that endovascular device 1016 makes several circumferential turns around scaffold 1028). Additionally or alternatively, endovascular device can be interwoven through scaffold 1028 such that endovascular device 1016 defines a zig-zag pattern (e.g., a skew apeirogon pattern, and/or having abrupt turns in different circumferential directions around scaffold 1028).

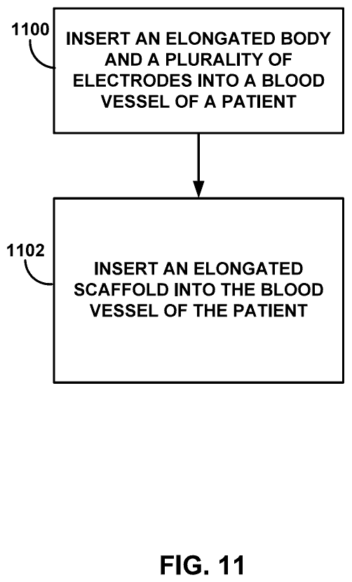

FIG. 11 is a flow diagram of an example method of placing an endovascular device into the vasculature of a patient, and is described with respect to endovascular device 16, electrodes 17, and elongated scaffold 28 of FIGS. 1 and 2, but is applicable to any of the endovascular devices and elongated scaffolds described in this disclosure. The technique of FIG. 11 includes inserting an elongated body (e.g., of endovascular device 16) and plurality of electrodes 17 carried by the elongated body of endovascular device 16 into a blood vessel of patient 12 (1100) and inserting an elongated scaffold 28 in the blood vessel of patient 12 (1102).

A clinician may observe, using a suitable medical imaging technique (e.g., fluoroscopic imaging), a portion of endovascular device 16 while distally advancing distal portion 15 of endovascular device 16 to a target location within the vasculature of patient 12. In some examples, inserting an elongated body of endovascular device 16 into vasculature of a patient (1100) includes initially introducing a guidewire, guide catheter, or another guide member into the vasculature of the patient to a target treatment site (e.g., within the neurovasculature, proximate one or more brain or other tissue sites). The elongated body of endovascular device 16 may then be introduced over the guidewire and advanced to the target treatment site. Additionally, or alternatively, endovascular device 16 may be introduced into vasculature of a patient with the aid of a guide catheter. For example, the guide catheter may be initially introduced into vasculature of a patient and positioned adjacent a target treatment site. Endovascular device 16 may then be introduced through an inner lumen of the guide catheter. In some examples, inserting elongated body of endovascular device 16 includes slightly pushing endovascular device 16 to position the elongated body and/or electrodes 17 towards the outer curve of the vessel. In some examples, inserting elongated body of endovascular device 16 includes slightly retracting endovascular device 16 to position the elongated body and/or electrodes 17 towards the inner curve of the vessel. Positioning endovascular device 16 and/or electrodes 17 towards and inner or outer curve of the blood vessel may depend on the location of interest for electrical stimulation therapy and/or sensing.

Inserting elongated scaffold 28 into the blood vessel of patient 12 (1102) may occur contemporaneously with, before, or after inserting endovascular device 16. In some examples, after advancing endovascular device 16 (e.g., distal portion 15) to the target treatment site, scaffold 28 is advanced distally through the vasculature and positioned adjacent a portion of endovascular device 16 (e.g., directly adjacent distal portion 15, including electrodes 17). In some examples, once endovascular device 16 with electrodes 17 have been delivery via a first delivery system, scaffold 28 is delivered via a second delivery system. In some examples, once positioned adjacent electrodes 17 and deployed (e.g., expanded), scaffold 28 exerts a radial outward force to urge electrodes 17 against a blood vessel wall. To exert the radial outward force, scaffold may be transformed from a delivery configuration to a deployed (e.g., expanded) configuration. Even in examples where scaffold 28 is configured to self-expand, the method can further include expanding the scaffold via a suitable expansion mechanisms (e.g., via a balloon, pullwire, etc.), such as when scaffold 28 is first delivered into the vasculature adjacent the endovascular device. In other examples, scaffold 28 is delivered contemporaneously with endovascular device 16 including plurality of electrodes 17, and scaffold is deployed (e.g., to provide a radial force to electrodes 17) once scaffold 28 and endovascular device 16 with electrodes 17 are positioned at the target treatment site. In still other examples, scaffold 28 is inserted into the vasculature before endovascular device 16 and electrodes 17. In some examples where scaffold 28 is inserted into the blood vessel of patient 12 before endovascular device 16 and electrodes 17, scaffold 28 is only deployed (e.g., to provide a radial outward force to urge endovascular device 16 and/or electrodes 17 against the vessel wall) after endovascular device 16 and electrodes 17 has been delivered to the target treatment site and positioned adjacent scaffold 28.

In some examples, where a plurality of scaffolds is provided (e.g., in a kit), the method can further include selecting a scaffold from the plurality of scaffolds, wherein each scaffold of the plurality of scaffolds is configured to have a different degradation profile (e.g., a different rate of degradation). In some examples, the scaffold is selected may be selected (e.g., by a clinician, or automatically) based on the one or more patient factors noted above, including one or more of a biological indicator (e.g., age, gender, etc.), a disease state (e.g., a disease that may otherwise affect blood composition, viscosity, etc.), an implant location (e.g., the specific blood vessel with in the neurovasculature), a blood vessel size (e.g., inner diameter), a blood vessel type (e.g., a vein versus an artery, wherein blood flow characteristics may differ between a vein and an artery as the walls of veins may be thinner and less elastic as compared to arteries), a blood pressure (e.g., which may vary from patient to patient, or even between time periods for a given patient), and/or a blood velocity.

In some examples, the method further includes delivering a therapy agent into the vasculature of the patient (e.g., via the scaffold 28, or by other means). In some examples, the therapy agent includes an anticoagulant agent, an antithrombosis agent, an antiplatelet agent (e.g., DAPT), or the like. The therapy agent may be delivered via bolus injection (e.g., injection into the vasculature), peroral delivery (e.g., pill or other oral medication), or by being released from endovascular device 16 and/or scaffold 28 (via a coating or via a material compounded with therapy agent, as described above).

In some examples, the method further includes repositioning or removing endovascular device 16 with electrodes 17 and/or scaffold 28. In examples of temporary therapy delivery (e.g., such as a trial period), or where endovascular device 16 and/or electrodes 17 need to be repositioned to improve electrical therapy delivery and/or sensing via electrodes 17, endovascular device 16 with electrodes 17 and/or scaffold 28 may be repositioned within the vasculature or removed from the vasculature, e.g., with the aid of a sheath, retrieval catheter, or another suitable mechanism.

In some examples, the method further includes delivering an agent (e.g., a liquid) configured to increase or decrease the degradation rate of scaffold (e.g., via an injection). It some examples, it may be desirable to speed up the degradation of the scaffold than would otherwise occur without intervention. For example, where a sufficient level of endothelization has occurred (e.g., as observed via a suitable medical imaging technique) and the scaffold is no longer needed to promote endothelization, a clinician can inject a fluid to increase the rate of degradation or resorption of the scaffold.

The operations and techniques described in this disclosure, including those attributed to system 10, medical device 14, programmer 20, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field-programmable gate array (FPGAs), or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include random-access memory (RAM), read-only memory (ROM), programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium includes a non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.
Example 1: An endovascular system includes an elongated body configured to be introduced into a blood vessel of a patient; a plurality of electrodes carried by the elongated body; and an elongated scaffold configured to be introduced into the blood vessel and configured to transform between a relatively low-profile delivery configuration and a deployed configuration, wherein in the deployed configuration, the elongated scaffold is configured to exert a radial force to urge the elongated body and the plurality of electrodes towards a wall of the blood vessel, wherein, after being introduced into the blood vessel of the patient, the elongated scaffold is configured to resorb or degrade over a period of time, wherein a degradation profile of the elongated scaffold over the period of time corresponds to a level of endothelization proximate the plurality of electrodes.
Example 2: The endovascular system of example 1, wherein the elongated scaffold is configured to maintain contact of the plurality of electrodes with the wall of the blood vessel for at least a portion of the period of time, and wherein the portion of the period of time where the elongated scaffold maintains contact of the plurality of electrodes with the wall of the blood vessel corresponds to a threshold level of endothelization proximate the plurality of electrodes.
Example 3: The endovascular system of example 2, wherein the elongated scaffold is configured to exert at least a threshold force on the plurality of electrodes for the portion of the period of time to maintain contact of the plurality of electrodes with the wall of the blood vessel.
Example 4: The endovascular system of any of examples 1 through 3, wherein the scaffold comprises a plurality of struts, wherein each strut of the plurality of struts comprises a first material and a second material, wherein the first material at least partially surrounds the second material, wherein the second material is configured to resorb or degrade faster than the first material.
Example 5: The endovascular system of any of examples 1 through 4, wherein the degradation profile of the elongated scaffold corresponds to one or more patient factors, wherein the one or more patient factors include a biological indicator, a disease state, an implant location, or a blood pressure.
Example 6: The endovascular system of any of examples 1 through 5, wherein the elongated scaffold is at least partially self-expanding.
Example 7: The endovascular system of any of examples 1 through 6, wherein prior to being introduced into the blood vessel of the patient, at least a portion of the elongated body is interwoven into the elongated scaffold.
Example 8: The endovascular system of any of examples 1 through 7, wherein the elongated scaffold comprises at least one of a braided stent, laser-cut tube, or a formed wire mesh.
Example 9: The endovascular system of any of examples 1 through 8, wherein the elongated scaffold comprises one or more of Polyglycolic acid (PGA), Polylactic acid (PLA), Polylactic-coglycolic acid (PLGA), Polycaprolactone (PCL), Polydioxanone (PDO), Polyethylene glycol (PEG), or Polytrimethylene carbonate (PTMC).
Example 10: The endovascular system of any of examples 1 through 9, wherein the elongated scaffold comprises one or more of Magnesium or a Magnesium alloy.
Example 11: The endovascular system of any of examples 1 through 10, wherein the elongated scaffold comprises a radiopaque material.
Example 12: The endovascular system of example 11, wherein the radiopaque material is barium sulfate.
Example 13: The endovascular system of any of examples 1 through 12, wherein when implanted, an elongated scaffold distal end extends distally of a distal-most electrode of the plurality of electrodes and an elongated scaffold proximal end extends proximally of a proximal-most electrode of the plurality of electrodes.
Example 14: The endovascular system of any of examples 1 through 13, wherein when implanted into the blood vessel, a portion of the elongated body positioned adjacent the elongated scaffold is configured to linearly extend between a proximal end of the elongated scaffold and a distal end of the elongated scaffold.
Example 15: The endovascular system of any of examples 1 through 13, wherein when implanted into the blood vessel, a portion of the elongated body positioned adjacent the elongated scaffold is configured to coil around the elongated scaffold between a proximal end of the elongated scaffold and a distal end of the elongated scaffold.
Example 16: The endovascular system of any of examples 1 through 15, wherein the elongated body and the elongated scaffold are configured to be delivered together via a delivery tool to a target location within vasculature of the patient.
Example 17: The endovascular system of any of examples 1 through 15, wherein the elongated body and the elongated scaffold are configured to be delivered separately to a target location within vasculature of the patient.
Example 18: The endovascular system of any of examples 1 through 17, further comprising processing circuitry configured to deliver electrical stimulation therapy to the patient or sense a patient parameter via the plurality of electrodes.
Example 19: The endovascular system of example 18, wherein the patient parameter includes an impedance detected via one or more electrodes of the plurality of electrodes, and wherein the processing circuitry is configured to detect a level of endothelization proximate the plurality of electrodes based on the impedance detected via the one or more electrodes of the plurality of electrodes.
Example 20: The endovascular system of any of examples 1 through 19, wherein the elongated scaffold is configured to release a therapy agent configured to reduce thrombosis.
Example 21: The endovascular system of example 20, wherein the elongated scaffold comprises an external coating comprising the therapy agent.
Example 22: The endovascular system of examples 20 or 21, wherein the elongated scaffold is configured to elute the therapy agent from within the elongated scaffold.
Example 23: The endovascular system of any of examples 20 through 22, wherein the therapy agent includes one or more of an anticoagulant agent or an antiplatelet agent.
Example 24: A method includes inserting, into a blood vessel of a patient, an elongated body and a plurality of electrodes carried by the elongated body; and inserting, into the blood vessel of the patient, an elongated scaffold, the elongated scaffold configured to transform between a relatively low-profile delivery configuration and a deployed configuration, wherein in the deployed configuration, the elongated scaffold exerts a radial force to urge the elongated body and the plurality of electrodes towards a wall of the blood vessel, wherein, after being introduced into the blood vessel of the patient, the elongated scaffold resorbs or degrades over a period of time, and wherein a degradation profile of the elongated scaffold over the period of time corresponds to a level of endothelization proximate the plurality of electrodes.

Various examples of the disclosure have been described. Any combination of the described systems, operations, or functions is contemplated. These and other examples are within the scope of the following claims.

What is claimed is:

1. An endovascular system comprising:
an elongated body configured to be introduced into a blood vessel of a patient;
a plurality of electrodes carried by the elongated body; and
an elongated scaffold configured to be introduced into the blood vessel and configured to transform between a relatively low-profile delivery configuration and a deployed configuration,
wherein in the deployed configuration, the elongated scaffold is configured to exert a radial force to urge the elongated body and the plurality of electrodes towards a wall of the blood vessel,
wherein, after being introduced into the blood vessel of the patient, the elongated scaffold is configured to resorb or degrade over a period of time,
wherein a degradation profile of the elongated scaffold over the period of time corresponds to a level of endothelization proximate the plurality of electrodes.

2. The endovascular system of claim 1,
wherein the elongated scaffold is configured to maintain contact of the plurality of electrodes with the wall of the blood vessel for at least a portion of the period of time, and
wherein the portion of the period of time where the elongated scaffold maintains contact of the plurality of electrodes with the wall of the blood vessel corresponds to a threshold level of endothelization proximate the plurality of electrodes.

3. The endovascular system of claim 2,
wherein the elongated scaffold is configured to exert at least a threshold force on the plurality of electrodes for the portion of the period of time to maintain contact of the plurality of electrodes with the wall of the blood vessel.

4. The endovascular system of claim 1,
wherein the scaffold comprises a plurality of struts,
wherein each strut of the plurality of struts comprises a first material and a second material, wherein the first material at least partially surrounds the second material,
wherein the second material is configured to resorb or degrade faster than the first material.

5. The endovascular system of claim 1,
wherein the degradation profile of the elongated scaffold corresponds to one or more patient factors,
wherein the one or more patient factors include a biological indicator, a disease state, an implant location, or a blood pressure.

6. The endovascular system of claim 1, wherein the elongated scaffold is at least partially self-expanding.

7. The endovascular system of any of claim 1,
wherein prior to being introduced into the blood vessel of the patient, at least a portion of the elongated body is interwoven into the elongated scaffold.

8. The endovascular system of any of claim 1, wherein the elongated scaffold comprises at least one of a braided stent, laser-cut tube, or a formed wire mesh.

9. The endovascular system of any of claim 1, wherein the elongated scaffold comprises one or more of Polyglycolic acid (PGA), Polylactic acid (PLA), Polylactic-coglycolic acid (PLGA), Polycaprolactone (PCL), Polydioxanone (PDO), Polyethylene glycol (PEG), or Polytrimethylene carbonate (PTMC).

10. The endovascular system of any of claim 1, wherein the elongated scaffold comprises one or more of Magnesium or a Magnesium alloy.

11. The endovascular system of claim 1, wherein the elongated scaffold comprises a radiopaque material.

12. The endovascular system of claim 11, wherein the radiopaque material is barium sulfate.

13. The endovascular system of claim 1,
wherein when implanted, an elongated scaffold distal end extends distally of a distal-most electrode of the plurality of electrodes and an elongated scaffold proximal end extends proximally of a proximal-most electrode of the plurality of electrodes.

14. The endovascular system of claim 1, wherein when implanted into the blood vessel, a portion of the elongated body positioned adjacent the elongated scaffold is configured to linearly extend between a proximal end of the elongated scaffold and a distal end of the elongated scaffold.

15. The endovascular system of claim 1, wherein when implanted into the blood vessel, a portion of the elongated body positioned adjacent the elongated scaffold is configured to coil around the elongated scaffold between a proximal end of the elongated scaffold and a distal end of the elongated scaffold.

16. The endovascular system of claim 1, wherein the elongated body and the elongated scaffold are configured to be delivered together via a delivery tool to a target location within vasculature of the patient.

17. The endovascular system of claim 1, wherein the elongated body and the elongated scaffold are configured to be delivered separately to a target location within vasculature of the patient.

18. The endovascular system of claim 1, further comprising processing circuitry configured to deliver electrical stimulation therapy to the patient or sense a patient parameter via the plurality of electrodes,
wherein the patient parameter includes an impedance detected via one or more electrodes of the plurality of electrodes, and wherein the processing circuitry is configured to detect a level of endothelization proximate the plurality of electrodes based on the impedance detected via the one or more electrodes of the plurality of electrodes.

19. The endovascular system of claim 1, wherein the elongated scaffold is configured to release a therapy agent configured to reduce thrombosis, and wherein the therapy agent includes one or more of an anticoagulant agent or an antiplatelet agent.

20. A method comprising:
inserting, into a blood vessel of a patient, an elongated body and a plurality of electrodes carried by the elongated body; and
inserting, into the blood vessel of the patient, an elongated scaffold, the elongated scaffold configured to transform between a relatively low-profile delivery configuration and a deployed configuration,
wherein in the deployed configuration, the elongated scaffold exerts a radial force to urge the elongated body and the plurality of electrodes towards a wall of the blood vessel,
wherein, after being introduced into the blood vessel of the patient, the elongated scaffold resorbs or degrades over a period of time, and
wherein a degradation profile of the elongated scaffold over the period of time corresponds to a level of endothelization proximate the plurality of electrodes.
